(19) United States
(12) Patent Application Publication
(10) Pub. No.: US 2022/0296689 A1
(43) Pub. Date: Sep. 22, 2022

(54) PARASITIC NEMATODE VACCINE

(71) Applicant: The University Court of the University of Edinburgh, Edinburgh Lothian (GB)

(72) Inventors: Amy Buck, Edinburgh (GB); Kyriaki Neophytou, Edinburgh (GB)

(21) Appl. No.: 17/638,542

(22) PCT Filed: Aug. 8, 2020
(86) PCT No.: PCT/GB2020/052079

§ 371 Date: Feb. 25, 2022

(30) Foreign Application Priority Data
Aug. 30, 2019 (GB) 1912528.5

(51) Int. Cl. A61K 39/00; C07K 14/435; A61P 37/04

(52) U.S. Cl. CPC A61K 39/0003; C07K 14/4354; A61P 37/04

(57) ABSTRACT

There is discussed nematode antigens capable of causing an immune response in a host such that a protective effect is provided to the host in relation to the nematode. Antigens, compositions for the treatment of parasitic nematode infections, methods of prophylaxis and treatment of parasitic nematode infections, and vaccines to reduce and/or control parasitic nematode infections are provided.

### FIELD OF THE INVENTION

The present invention relates to nematode antigens capable of causing an immune response in a host such that a protective effect is provided to the host in relation to the nematode. In particular the present invention relates to antigens, compositions for the treatment of parasitic nematode infections, methods of prophylaxis and treatment of parasitic nematode infections, and vaccines to reduce and/or control parasitic nematode infections.

### BACKGROUND OF THE INVENTION

Parasitic nematodes infect a quarter of the world's human population and gastrointestinal nematodes in particular rank first in terms of the global health burden of the neglected tropical diseases. Gastrointestinal nematodes are also highly prevalent in livestock, with implications to economic viability and food security in Europe and world-wide.

There is a limited range of anthelmintic drugs, which show mounting resistance and do not prevent reinfection. Additionally, there are presently only two commercially available vaccines which prevent infection by parasitic nematodes, both of which have significant limitations as described below.

Human gastrointestinal nematode infections are a huge issue for human health in the developing world and for economic viability and food security across the globe. Worldwide, an estimated 600 million people carry hookworm infections, a neglected tropical disease (NTD) leading the list in terms of Years Lived with Disability (1.6 Million YLDs) caused by NTDs. Hookworm affects the poor, resulting in impaired physical and cognitive development in children and a 40% reduction in future wage-earning. Current treatments are typically with the medications albendazole or mebendazole for one to three days. Reinfection rates after treatment are however extremely high, with up to 80% of patients being re-infected within 30-36 months.

One of the contributing factors towards chronic parasitic nematode infection is that a number of parasitic nematodes are capable of masking their presence, avoiding expulsion or inducing tolerance using immunomodulatory behaviour. The full range of mechanisms is not yet known, however such nematodes release extracellular vesicles (EVs) into their environment, and it has been shown that targeting EVs by vaccination can provide protective immunity against a helminth parasite (Coakley et al, Extracellular Vesicles from a Helminth Parasite Suppress Macrophage Activation and Constitute an Effective Vaccine for Protective Immunity. Cell Reports (2017). 19:1545-1557). It would be advantageous to provide alternative vaccine strategies to nematode parasite infections.

### SUMMARY OF THE INVENTION

The inventors have determined a target protein which can be provided to a host or potential host of a parasitic nematode, wherein the target protein has the ability to promote an immune response such that the host will reduce, prevent, or minimise infection, i.e. provide a response that is protective. Without wishing to be bound by theory, it is considered the target protein is associated with a class of immunomodulatory molecules released by a parasite, wherein the immunomodulatory molecules actively condition the host environment to promote survival of the parasite. Suitably, the target protein may function in the host to modulate the intestinal environment to promote parasite survival.

The argonaute protein family (AGO) is characterised as a family of proteins which comprise a PIWI domain and a PAZ domain that bind small RNAs (siRNAs). A particular Argonaute (exWAGO (—extracellular Worm AGO)) has been determined to be released by the gastrointestinal nematode Heligmosomoides bakeri, which whilst clearly an argonaute protein by the presence of PIWI and PAZ domains and its ability to bind small RNAs, is restricted to nematode lineages and is very distinct from mammalian argonautes (less than 27% identity with human argonautes). Previous work by the inventors determined that the specific Argonaute protein (exWAGO) is secreted in parasite EVs and that it may constitute a key mechanism for directing the selective packaging and export of specific parasite siRNAs in EVs for subsequent delivery to host cells (Chow et al., “Secretion of an Argonaute protein by a parasitic nematode and the evolution of its siRNA guides”, Nucleic Acids Research, 2019, 47). The exWAGO protein was determined via ultracentrifugation and sucrose gradient experiments to co-purify with EVs and such exWAGO was determined to be protected from proteinase K degradation when sucrose purified EV fractions were treated with proteinase K, but became susceptible when the EVs were lysed by detergent. As the exWAGO was ‘protected’ from degradation, it was considered that the exWAGO would not be immune accessible and thus would not give rise to an immune response when functioning in a host. Surprisingly, the inventors have determined that recombinant exWAGO can be used to induce an immune response against functionally active exWAGO in the host and that such an immune response can be protective against the parasite such that the host can effectively be vaccinated against infection by the parasite. Whilst not wishing to be bound by theory, the inventors consider the protective immune response is blocking the functional activity of the exWAGO. Whilst an example of the target protein discussed herein is from the parasite Heligmosomoides bakeri (a model intestinal round worm of rodents that has also been called Heligmosomoides polygyrus in the literature) this target protein is conserved across a range of parasitic nematodes and appears to function in the same way in a range of parasite nematodes, for example in view of its abundant expression in related parasites. Consequently, it is considered the target protein from Heligmosomoides bakeri or variants, fragments, homologues or orthologues thereof which provide an immune response could be utilised as a vaccine against parasitic infections in humans (e.g. hookworms—Necator americanus), or livestock (e.g. sheep/goat brown worms: Teladorsagia circumcinta). As would be understood, livestock may further include cattle, horses and camelids. It is considered that cattle, equine and Camelid nematode pathogens which would be similar to Clade V exWAGO family (Nematodirus) or Clade III exWAGO family members as discussed herein would also be modulated by an immune response generated by exWAGO as discussed herein, in particular SED ID NO: 1 exWAGO from H bakeri. Further, it is considered that it may also provide a protective effect in domesticated animals (e.g. dog heart worm—Dirofilaria immitis).

The inventors consider that use of argonaute protein of a nematode lineage as a recombinant vaccine would allow associated parasitic immunomodulatory molecules (RNAs) to be blocked. Moreover, due to the conservation between argonautes of a nematode lineage, it is considered that antibodies to exWAGO proteins generated against a first parasite argonaute protein, for example Heligmosomoides bakeri, may be effective in the treatment of parasitic infections in other animals for example in cattle, sheep, equids, camelids, dogs, humans, in particular against Necator americanus (human hookworm), Teladorsagia circumcinta, Haemonchus contortus and Nematodirus (sheep parasites), Dirofilaria immitis (dog heartworm), and Ostertagia ostertagi (cattle parasite).

The use of recombinant exWAGO protein to provide immunomodulation such that a protective response is formed against parasite infection is surprising, as it was previously understood that functioning exWAGO, providing selectivity of RNA export, was not accessible in view of its association with the EV. As such, it was understood the exWAGO would be hidden from molecules of the immune system and thus recombinant exWAGO would not have elicited an immune response capable of blocking the functionality of exWAGO in a host. Moreover, it is understood that Argonaute proteins have not previously been suggested as vaccine candidates in any model systems.

Accordingly, a first aspect of the present invention provides an exWAGO protein or fragment thereof from a parasitic nematode or a nucleic acid sequence capable of expressing exWAGO or a fragment thereof for use in the treatment of parasitic nematode infection. As noted it is considered a protective immune response can be provided in a host animal to reduce infection of the host animal by parasitic nematodes. Suitably an immune response may elicit a response that results in a decrease in parasitic burden of at least 30%, at least 40%, at least 50%, at least 60%, at least 70%, at least 80%, at least 90% in a host in relation to a non-treated/non-vaccinated host or suitable control (adjuvant with no antigen). An exWAGO protein as discussed herein may suitably be provided by any recombinant system. Suitably a recombinant system to provide exWAGO may be provided in insect cell expression systems].

Suitably an exWAGO protein may comprise a PIWI domain and a PAZ domain and be capable of binding RNA.

Suitably an exWAGO protein may comprise a sequence of SEQ ID NO: 1 (exWAGO released by Heligmosomoides bakeri) or have a sequence identity of at least 60% to SEQ ID NO: 1.

SEQ ID NO: 1, H. Bakeri exWAGO protein sequence:
MDQLKTGMGQLSVGAVALPEKRSPGGIGNKVDFVTNLTELSLKPNVPYYK
YDIRMYIVYKGNDALEHLKELTKQTKDDFPEQERKSAAVAVYKHLCKTYK
DVFLPDGALLYDRAAVLFSAQRQLKLDGEEKQFMLPASVVSSAGPDATGI
RVVIKKVKDQFQVTSNDLSKAVNVRDMERDKGILEVLNLAVSQKGYMETS
QFVTYGSGVHYLFDHRALGFRDNELPELMDGKYMGIGLTKSVKVLEGDSG
KGNSAFVVTDVTKGAFHVDEQNLMEKISQMSIFFDQRTGQSSFNAKNAMQ
PFNQKAILQQIKGLYVRTTYGKKKTFPIGNLAAAANALKFQTADGAQCTV
EQYFKKHYNIQLKYPGMFTVSERHNPHTYYPVELLTVAPSQRVTLQQQTP
DQVASMIKASATLPQTRLHQTKIMKDALDITPRNHNLATAGISVANGFTA
VSGRVLPSPRIAYGGNQILRPVDNCKVVNGDRSVFLEPAKLTNWAVCVTL
TQQDARRLQIKEYISRVEMRCRNRGMQVDPVAEVFTLKHQTFDGLKEVVY
ASQKQKNRRYLMFITSDGIKQHDSIKLLEVEYQIVSQEIKGSKVDAVVTK
NQNQTLDNVVAKINMKLGGVNYNVMLGVKNDDKAFSVVLNDKDRMFVGFE
ISNPPALSKVEIERGASYKMPSVLGWGANCAGNHQQYIGDYVYIQPRQSD
MMGAKLSELIVDILKRFRAATTIAPRHIVLYFSGISEGQFSLVTDTYMRA
VNTGIASLSPNYKPSVTAVAVSKDHNERIYKTNISGNRATEQNIPPGTVI
DTKIVSPVINEFYLNSHSAFQGTAKTPKYSLLADNSKIPLDVIEGMTHGL
CYLHEIVTSTVSVPVPLIVADRCAKRGHNVYIANSNQGEHSVNTIDEANA
KLVNDGDLKKVRYNA

Suitably an exWAGO protein may comprise a sequence of SEQ ID NO: 1 (exWAGO released by Heligmosomoides bakeri) or have a sequence identity of at least 65%, at least 70%, at least 75%, at least 80%, at least 85%, at least 90%, at least 92%, at least 95%, at least 97%, at least 99% to SEQ ID NO: 1.

Suitably the exWAGO protein may be a homologue of SEQ ID NO: 1. Suitably the exWAGO protein may be a paralogue of SEQ ID NO: 1. Suitably the exWAGO may be an orthologue of SEQ ID NO: 1. Suitably, the homologue, paralogue or orthologue are functionally similar to the protein comprising the amino acid sequence of SEQ ID NO:1. Suitably the exWAGO may be a homologue of a Clade or Clades associated with nematodes.

Argonaute proteins have been classified, based on sequence identity, into several clades. A BLAST sequence alignment of H. bakeri exWAGO protein sequence SEQ ID NO: 1, against the human genome showed that the closest protein sequence in the human genome is human argonaute 1 protein, at 27% identity and 46% positive similarity. Orthologues were identified in Clade III and Clade V nematode worms, showing that the sequence of exWAGO is highly conserved amongst Clade V and to a lesser extent Clade III nematode worms, with sequence alignment of H. bakeri exWAGO protein sequence against the orthologues in N. americanus (SEQ ID NO: 2) and T. circumcinta (SEQ ID NO: 3) showing 77% identity and 87% similarity, and 82% identity and 90% similarity, respectively. This therefore shows that exWAGO is highly conserved in Clade V nematode worms.

Clade V is defined as and Clade III as defined by genomic analyses, for example as discussed in, Comparative genomics of the major parasitic worms, 2019. Nature Genetics 51, 163-174.

exWAGO N. americanus
SEQ ID NO: 2
MADQLKKRMGELTVDTVALPEKRAPGTLGAATEFVTNLTSLKLKPNVPFF
KYDIRMYIVYKSSDGKEHLKELTKQTKDDFPEQERKTGTVLVYKHLLKTN
PSVFPQDGALLYDRAAVLFSAQKQIKLDGEEKVFMLPASLVPSAGEDATG
VRVVVKKVTEGFQVTSNDLAKAVNVRDFEKDKGILEVLNLAVSQKGYMET
SQFVTYGSGVHYLFDHRALGFRESGLFKIIKLLFKIIGLXXXVKVLEGEG
ESCTAYVVTDVTKGAFHIDDQNLLEKISQMSMFIDPRSGQSHFNIQAAMQ
PINQKNILQL1KGLYVRTTYGKKRTFPIGNIAQAANQLKFQTVEGTQCTV
EQYFKKHYNIVLKHPGMFTVSERHSPHTYYPVELLRVAPSQRVTLQQQTP
DQVATMIKACATLPQNRLHQTKLLKDALDIKPGNPRLAVAGISVENGFTT
VPGRVLPPPSIIYGGNQLVKPIDNCKWNGDRSRFLEPARLYNWAVCATLT
PNDSRRLHIKEYIVRVEGRCRQRGMDVEPCSEIFNLQRQNFESLKEVVYA
SQKEKDRRYLMFITSDHIKQHDLIKLLEIEYQIVSQEIKGSKVDAVLTRN
QNQTLDNVIAKINEKLGGVNYNIMLGSSPSDKANKWLYEKDRMFVGFEIS
NPPALSKAEIERGAAYKMPSVLGWGANCAKNPQQYLGDYVYIEPRQTDMM
GAKLSEIIVHILKRFRAATDVAPRHIVLYFSGISEGQWSLVADTYMRAIQ
TGIKSLSATYGPSLTALTVSKDHIERIYKSNITGNRATEQNIPPGTVVDT
KIVSPVINEFYLNAHSAFQGTTKTPKYALVYDDSNIPINAVEGMTHGLCY
LHEIITATVSMPVPLIVADRCAKRGHNVYIANSSQRNAVSCIKEANEKLV
NQGALQKVRYNA
exWAGO T. circumcinta
SEQ ID NO: 3
MADQLSGGMGKLSVAAVALPEKRAPGSLGTKLDFVTNLTGIKLKPNVPYY
KYDVRMYIVYKGNDGREVLKELTKQTKDDFPEQERKMAAVAIYKHLVKSY
KDIFPQDGQFFYDRAAVLFSAQREMKLGGPEKVITLPASLSPTAGSDAAG
IRVVIKKVTDGYQVTSNDLMKAVNVRDCERDKGILEVLNLAVSQKGYMET
SQFVTYGTGVHYLYDHRALGFRDNELPDLMDGKYMGIGLTKAVKVLEGDQ
GKSASAFVVTDVTKGAFHIDEQNLLEKISQMSIFFDPRTGQSTFSVKAAM
QPHNMKSILQLIKGLYVRTTYGRKRTFPIGNLAAAPNALKLQTSDGVQCT
IEQYFKKQYNVQLKYPGLFTVSERHNPHNYYPVELLTVAPSQRVTLQQQT
PDQVASMIKASATLPSNRLHQTKVMKEALDITPRNAKLASAGINVEDGFT
TVPGRVLPTPTILYGGSQTLKPVDNCKWNGDRSRFLEPAQLTNWAVCATL
TQNDARRLQIKDYVARVESRCRAKGMQVEAAAEIFTLTKQNFDGLREFYA
AQKKKNRKYLLFITSDGIKQHDLIKLLEVEYQIVSQEVKGSKVDSVMFKN
QNQTLDNVIAKINMKLGGVNYNVVLGSKPNDPASKWLNDKDRLFVGFEIS
NPPALSKMEIERGATYKMPSVLGWGANCAANPQHYIGDYVYIKPRQSDMM
GAKLSELIVEILKKFRGATSLAPRHIVLYFSGISEGQFSLVTDTYMKAIN
TGITSLSANYRPSVTALAVSKDHNERLYKSNISGSRANEQNIPPGSVVDT
KIVSPVINEFYLNSHSAFQGTAKTPKYSLLADDSKIPLDVIEGMTHGLCY
LHEIVTSTVSVPVPLIVADRCAKRGHNIFIANSNLGSAAVSSIEEANEKL
VNHGELEKVRYNA

Suitably the exWAGO protein for use in the invention may be classified as Clade V. Suitably the exWAGO protein for use in the invention may be classified in Clade III.

Suitably the exWAGO protein may comprise a sequence of SEQ ID NO: 1 or be an orthologue to SEQ ID NO: 1. For example an orthologue may have the same secondary or tertiary structure to the amino acid sequence comprising SEQ ID NO: 1. Suitably the orthologue may have a sequence identity in the range of 30% to 40%, 41% to 50%, 51% to 60%, 61% to 70%, 71% to 80%, 81% to 90%, at least 91% to SEQ ID NO: 1.

Given the high expression of the exWAGO protein in other parasitic worms, for example the sheep parasite Teladorsagia circumcincta and the human hookworm Necator americanus, and the conservation between these proteins, but not to host (for example mammalian) Argonautes, it is considered argonaute proteins from nematode parasites can be distinguished from host Argonautes. The parasite exWAGO proteins may therefore be selected as vaccine targets either for the specific parasite or to generate an antibody that recognises the parasite protein of related parasites.

Based on published data it would have been expected that functional exWAGO operates solely within vesicles and would not thus be expected to be accessible to host antibodies as vesicular exWAGO was not considered to be surface-accessible. Thus, prior to the further work discussed herein, exWAGO would not be considered as a viable recombinant target for modulating immune response in the host. Surprisingly, the present inventors discovered that recombinant exWAGO protein is able to induce an antibody response and significantly reduce parasitic nematode worm survival when delivered to a subject in need thereof. Without wishing to be bound by theory, this indicates that exWAGO is accessible to antibodies. Suitably antibodies may target vesicular exWAGO, vesicle-free exWAGO and/or vesicular associated exWAGO, in a previously unrecognised manner to functionally modulate the effect of exWAGO or an environment exposed exWAGO on the parasite.

Suitably an exWAGO protein or fragment or a nucleic acid capable of encoding an exWAGO protein or fragment thereof may elicit an immune response in an animal that is capable of providing a protective effect to the animal against infection/infestation of a nematode parasite. This protective effect may enable a reduction in the parasitic burden in a host animal. Suitably a reduction in parasitic burden may be observed by a decrease in the parasite eggs provided in host faecal matter—host faecal egg count. Suitably a reduction in parasitic burden may be observed by a decrease in the number of adult worms present in the host. Suitably a reduction in parasitic burden may be a decrease in parasitic larval development. Parasitic burden in a subject provided with exWAGO may be assessed relative to a subject that has not been provided with exWAGO prior to infection with the parasite. Suitably, either additionally or alternatively blocking exWAGO may prevent a parasite from modulating host and improve host health, for example by blocking nutrient absorption by the parasite to blocking another pathway which leads to decreased health of the host through the parasitic RNAs targeting host genes.

It will be understood by those of skill in the art that it may not be required to utilise the entire amino acid sequence of exWAGO, for example of SEQ ID NO: 1 to elicit a suitable immune response to provide a protective response against the parasite from which the exWAGO originates. The present invention also comprises antigenic fragments and their use in compositions for immune system modulation, or vaccine preparations. Suitably a fragment of exWAGO may be an amino acid sequence comprising at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600 amino acids or n-1 wherein n is the size n of the exWAGO amino acid encoding the complete exWAGO protein. Suitably the fragment may be a C-terminal fragment or an N-terminal fragment or a fragment selected by epitope scanning, deletion of amino acids at the N or C terminus, or internally within the molecule. Suitably a fragment may be hydrophilic in nature.

Suitably, variants of exWAGO may be provided wherein the variants are at least 60%, 65%, 70%, 75%, 80%, 85%, 90%, 95%, 97%, or 99% identical to SEQ ID NO:1. The identity is with reference to the portion of amino acid sequence that corresponds to the SEQ ID NO: 1 sequence or portion thereof in a suitable alignment window taking into consideration the size of the variant, i.e. not including additional elements as discussed below. Suitably a variant may comprise from about 5 to about 10, 5 to about 20, 5 to about 30, 5 to about 40, 5 to about 50 amino acids which are substituted from the SEQ ID NO: 1 sequence. Suitably, the substituted amino acids may be conservative substitutions. Suitably variants may have additional amino acids to SEQ ID NO:1, for example a His-tag for example to facilitate protein isolation. Suitably variants may be modified either by conserved or non-conserved substitutions, insertions or deletions to modify the solubility or stability of the protein, for example to introduce or remove cleavage sites within the amino acid sequence. The variants may be a result of natural variation between species (orthologues) or introduced using recombinant methods. As will be appreciated where exWAGO is provided for use in the invention by expression of a nucleic acid sequence, for example a nucleic acid sequence encoding SEQ ID NO:1 or a fragment thereof, the nucleic acid sequence may be varied such that it still encodes the amino acid sequence, but utilises the redundancy of the genetic code to aid expression, for example to optimise codon expression, to aid insertion of the nucleic acid sequence in a vector, modify transcription or promotor or another regulatory sequence of the nucleic acid or the like. Suitably the nucleic acid variants may display about 50% to 100% sequence identity, about 65% to 100% sequence identity, about 75% to 100% sequence identity, about 80% to 100% sequence identity, about 90% to 100% sequence identity to a nucleic acid sequence encoding exWAGO in a parasitic nematode, for example encoding SEQ ID NO: 1.

According to a second aspect of the present invention there is provided a composition comprising an exWAGO protein or a fragment thereof or a nucleic acid sequence capable of expressing exWAGO as discussed in relation to the first aspect of the invention and optionally an adjuvant for use in the treatment or prophylaxis of a parasitic nematode.

Suitably a composition may comprise at least a second parasitic antigen to which an immune response may be raised. For example a second antigen may be an immunogenic component or components which elicits an immune response during natural infection, for example an amino acid sequence, nucleic acid sequence, carbohydrate or the like. Suitably the composition may further comprise an additional pharmaceutical excipient, or pharmaceutically active molecule. The second antigen may be distinct from exWAGO.

Advantageously, a composition of the second aspect of the invention may be used to raise an immune response or provide an immune response such as to vaccinate a subject in need thereof against parasitic nematode infection, in particular Clade III or Clade V parasitic nematode infection. Advantageously, this may prevent the need for the application of helminthicidal drugs and/or minimise the risk of re-infection and the spread of disease.

In embodiments, an exWAGO protein or fragment thereof or a nucleic acid sequence capable of expressing exWAGO from a first parasitic nematode may be used to promote or raise an immune response against a parasitic nematode of the same type. It will be understood that typically to elicit a suitable immune response the exWAGO antigen will be used from the species of interest. However, this may not always be the case. Cross species exWAGO antigen may be used to elicit an immune response i.e. exWAGO antigen from a parasitic nematode that targets a mouse may be used to vaccinate/modulate the immune response in a sheep, in cattle, horse, camelid, dog or human. The ability to confer protection against invasion and parasitisation of a host by a parasite following treatment can be determined by considering parasite burden in treated and non-treated subjects. Suitably, the immune response may prevent the occurrence of a further infection (subsequent or secondary infections) of the first parasitic nematode. In embodiments, an exWAGO protein or fragment thereof or a sequence capable of expressing exWAGO from a first parasitic nematode may be used to promote or raise an immune response against a second parasitic nematode. For example a second parasitic nematode may be from another genera. In embodiments, a first parasitic nematode may be a Clade III parasitic nematode and this can be used to promote an immune response against a different Clade III parasitic nematode.

In embodiments, a first parasitic nematode may be a Clade V parasitic nematode and this can be used to promote an immune response against a different Clade V parasitic nematode.

In embodiments, a first parasitic nematode may be a Clade III parasitic nematode and this can be used to promote an immune response against a Clade V parasitic nematode.

In embodiments, a first parasitic nematode may be a Clade V parasitic nematode and this may be used to promote an immune response against a Clade III parasitic nematode.

According to a third aspect of the present invention there is provided a method of raising an immune response against a parasitic nematode in an animal said method comprising the step of administering to the animal an exWAGO protein or fragment thereof from a parasitic nematode or a nucleic acid sequence capable of expressing exWAGO or a fragment thereof sufficient to induce an immune response in the animal against the parasitic nematode.

Suitably this provides a method of vaccinating a subject in need thereof against parasitic nematode infection, in particular infection of parasitic nematodes of Clade V. Suitably the vaccine comprises an exWAGO antigen or at least two antigens wherein one of the antigens comprises exWAGO antigen, and optionally at least one adjuvant (for example an adjuvant as discussed herein). In embodiments the adjuvant can be an aluminium based adjuvant. Suitably the adjuvant may be a lipid based adjuvant. Suitably a first aluminium and a second lipid based adjuvant may be utilised in combination.

According to a fourth aspect of the present invention there is provided exWAGO protein or a fragment thereof from a parasitic nematode or a nucleic acid sequence capable of expressing exWAGO or a fragment thereof for use in the treatment of or prevention of infection of parasitic nematode.

According to a fifth aspect of the present invention there is provided the use of exWAGO protein or a fragment thereof from a parasitic nematode or a nucleic acid sequence capable of expressing exWAGO or a fragment thereof in a method of manufacturing a medicament for use in the treatment of or prevention of infection of a parasitic nematode.

Suitably a recombinant exWAGO amino acid sequence may be optimised to provide increased expression in a recombinant vector. For example, codon optimisation may be utilised to enhance the efficiency of protein expression.

Suitably the animal may be human, sheep, goat, (or other ovine), bovine, equid, equine, camelid, camel or dog.

According to a sixth aspect of the present invention there is provided a method to determine and optionally select an epitope or antigenic fragment of exWAGO to provide antibodies or aptamers thereto wherein the antibodies or aptamers provide a protective immune response to a host subsequently infected with a parasitic nematode.

As discussed herein, exWAGO may have at least at 60% sequence identity to SEQ ID NO: 1. Suitably the exWAGO may be provided with an EV, associated to an EV or separate from an EV in an infected host. Suitably the exWAGO amino acid sequence or part thereof may be exposed on the surface of a vesicle. Suitably the exWAGO may be releasable from a vesicle in a parasitic nematode infected host.

As will be appreciated in the art, once an antigenic protein has been identified (as is the case of the present invention with exWAGO), standard techniques such as protein microarrays (e.g ELISPOT or ELISA) or high-throughput mutagenesis may be used to rapidly identify epitopes of that protein. Identified epitopes may be used as fragments to provide recombinant exWAGO. Structure-activity relationship (SAR) assays of the epitope(s) using the same high throughput techniques may then be used to design antibodies, binding fragments thereof or aptamers of that epitope. Such techniques are well known in the art. Suitably, antibodies, binding fragments thereof, aptamers, or peptides directed to structures—for example to bind to conserved regions of the Argonaute-PMID: 26351695 may provide separate aspects of the present invention. Suitably, an assay to determine small molecules, antibodies, binding fragments thereof or aptamers which bind to exWAGO and block its ability to bind to immunomodulatory factors, for example RNA, can be used to determine therapeutic molecules which block the function of exWAGO. Such an assay and molecules determined by this assay form a separate aspect of the present invention.

Adjuvants are well known in the art, and may be used to boost, alter or modify the immune response to the provision of a vaccine to a subject in need thereof. Suitably, the adjuvant may enhance the efficacy of the immune response raised or of a vaccine by increasing the immunogenic response to the exWAGO protein or fragment thereof, for example provided as a vaccine. Suitably, the adjuvant may modify the immune response to particular types of immune system cells: for example, by activating B cells instead of T cells depending on whether a humoral (antibody-based) immunity or cell-mediated immunity is intended. In some embodiments, the target Clade III or Clade V nematode worm can be a nematode worm which inhabits the gastrointestinal tract, and a humoral immune response is particularly advantageous. Suitably, the adjuvant may comprise a therapeutically effective amount of an adjuvant selected from a list comprising alum, aluminium hydroxide and aluminium phosphate, potassium sulphate, virosomes, lipid particles, lipid derivatives, mineral oil emulsions, Freund's complete adjuvant, squalene or paraffin oil or the like as would be understood in the art, for example as discussed at https://www.ema.europa.eu/en/documents/scientific-guideline/guideline-adjuvants-vaccines_en.pdf and https://www.cdc.gov/vaccinesafety/concerns/adjuvants.html.

Suitably, the composition may comprise two or more adjuvants. Suitably the protein may be provided with lipid encapsulation. Suitably, the composition of the present invention may be configured to elicit either an IgG or IgA response. Suitably the composition may involve oral delivery of bacterial spores, for example as discussed Wuttipong Phumrattanaprapin, Sujittra Chaiyadet, Paul J Brindley, Mark Pearson, Michael J Smout, Alex Loukas, Thewarach Laha, Orally administered Bacillus spores expressing an extracellular vesicle-derived tetraspanin protect hamsters against challenge infection with carcinogenic human liver fluke, The Journal of Infectious Diseases, jiaa516 wherein an oral vaccine was provided based on recombinant Bacillus subtilis spores expressing the large extracellular loop (LEL) of O. viverrini tetraspanin-2 (Ov-TSP-2) (a protein that is abundant on the surface of O. viverrini secreted extracellular vesicles).

Suitably, the target nematode worm may inhabit the gastrointestinal tract, alternatively the target nematode worm may inhabit the skin, lymph system, heart, lungs or brain. Suitably, immune modulators or vaccines which target nematode worms which inhabit the gastrointestinal tract may be configured to elicit a strong B-cell mediated response.

Suitably, vaccines or immune modulators which target nematode worms which inhabit the skin, lymph system, heart, lungs or brain may be directed towards a strong T-cell mediated response.

Suitably, the exWAGO protein or fragment thereof or a nucleic acid encoding an exWAGO protein of the first aspect of the invention may be used to raise an immune response against a parasitic nematode infecting mammals, suitably a Clade III or Clade V parasitic nematode worm. Suitably the parasitic nematode exWAGO protein or fragment thereof or nucleic acid encoding an exWAGO protein of the first aspect of the invention may be used to raise an immune response against a parasitic nematode of the order Strongylida, Rhabditida or Spirurida. Suitably an exWAGO protein or fragment thereof or nucleic acid encoding an exWAGO protein may be used to raise an immune response against at least one parasitic nematode selected from:

Nippostrongylus brasiliensis (rodents)

Ancylostoma caninum

Ancylostoma ceylanicum

Ancylostoma duodenale

Oesophagostomum dentatum

Angiostrongylus costaricensis

Dictyocaulus viviparous

Symphacia muris (rodents)

Enterobius vermicularis

Anisakis simplex

Ascaris suum

Ascaris lumbricoides

Thelazia callipaeda

Brugia malayi

Wuchereria bancrofti

Onchocerca flexuosa

Onchocerca ochengi

Acanthocheilonema viteae (rodents)

Suitably an immune response may be raised against parasitic nematodes of Clade V and Clade III, for example wherein Clade V includes:
Strongylida
Rhabditida
Diplogasterida and Clade III includes:
Oxyurida
Spirurida
Rhigonematida
Ascaridida,
   suitably against parasitic nematodes of Clade V or Clade III.

In embodiments, the parasitic nematode can be selected from a group comprising of Heligmosomoides polygyrus, Heligmosomoides bakeri, Necator americanus, Teladorsagia circumcincta, Dirofilaria immitis, Onchocerca volvulus, Loa loa, Haemonchus contortus, Teladorsagia circumcincta and Angiostrongylus cantonensis, Ostertagia ostertagi and Nematodirus

In embodiments, there is provided a peptide with at least 70%, at least 75%, at least 80%, at least 85%, at least 90%, at least 95%, at least 97%, at least 99% sequence identity with any one of SEQ ID Nos. 1 to 3 or fragments thereof as discussed herein. Suitably, the peptide comprise at least 80%, at least 90%, at least 95%, sequence identity with any two of SEQ ID Nos. 1 to 3 or fragments thereof as discussed herein.

Suitably the peptide may further comprise modifications, for example additional motifs to permit solubilisation, isolation, purification or digestion of the peptide. Such additional motifs are well known in the art, e.g. His-tag, thrombase cleavage site, (SGGGG)₃ solubility tag etc.

Suitably, the peptide may be adapted to increase its immunogenicity and/or stability. Suitably, the peptide may be adapted by being polyvalent, optionally bivalent or trivalent. In such embodiments, the peptide or aptamer can be linked by a linear or branching linker motif.

Suitably, the exWAGO protein or peptide may be synthetic or recombinant. Suitably the peptide may be provided by either partial or complete biosynthesis by yeast or bacteria. Synthetic and biosynthetic production of proteins or peptides are well known in the art. Such techniques dramatically reduce the manufacturing costs, ethical, production, safety, purity, and dosage issues associated with anti-helminth vaccine production in live hosts.

Suitably, the composition is provided for simultaneous, separate or sequential administration with one or more pharmaceutical active agents (a co-therapy) for the treatment or prophylaxis of parasitic nematodes, in particular Clade III or Clade V parasitic nematodes. Suitably, the pharmaceutical may be a helminthicide. Suitably the pharmaceutical may be selected from a group comprising ivermectin, pyrantel pamoate, benzimidazoles such as albendazole or mebendazole; levamisole, macrocyclic lactones, amino acetonitrile derivatives, spiroindoles, melarsomine, thiacetarsamide, milbemycin or selamectin. Suitably, the composition is provided for simultaneous, separate or sequential administration with one or more additional antigens, for example to add to an additional vaccine to provide a divalent or multivalent vaccine.

Accordingly, to a further aspect of the present infection there is provided a kit for eliciting a protective immune response to a parasitic nematode, in particular to a Clade III or Clade V parasitic nematode in a subject in need thereof, wherein the kit comprises an exWAGO protein as discussed herein and a further pharmaceutical active and/or adjuvant.

The subject to be treated using the exWAGO protein or fragment thereof or nucleic acid encoding the same as described herein may be a human. In alternative embodiments, the invention provides for the veterinary treatment of non-human mammals, for example cats, dogs, livestock including cows, sheep and goats.

Pharmaceutical compositions for use in accordance with the present invention may be formulated in a conventional manner for use in human and veterinary medicine using one or more pharmaceutically acceptable carriers or excipients.

Thus, the composition of the present invention may be formulated for oral, parenteral, depot or rectal administration or in a form suitable for administration by inhalation or insufflation (either through the mouth or nose), buccal, topical, transdermal or the like based on the target parasitic nematode being treated, in particular based on the Clade III or Clade V parasitic nematode being treated, and the type of immune response that the composition is intended to elicit. For instance, a gastrointestinal worm may be targeted by oral administration to trigger a humoral response, optionally where the composition is adapted to trigger a B-cell mediated response dictated by the type of adjuvant used.

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g.

lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Oral preparations are particularly preferred where a simple method of provision of the composition is desirable compared to more complex techniques such as parenteral techniques such as intravenous or intramuscular supply. For instance, for treatment of subjects in areas with a relatively low medical training such as in remote or rural areas. Preparations for oral administration may be suitably formulated to give controlled release of the active compound, e.g. to preferentially release in the duodenum or in the long intestine, and not in the stomach where compositions comprising peptide bonds may be digested.

Suitably, the composition may be configured for use orally.

Suitably, the composition may be configured for use by parenteral administration by bolus injection or continuous infusion.

Formulations for injection may be presented in unit dosage form e.g. in ampoules or in multi-dose containers, with an added preservative.

In some embodiments, the composition may be provided as a suspension, solution or emulsion in oily or aqueous vehicles, and may further comprise formulatory agents such as suspending, stabilising and/or dispersing agents. Alternatively, the composition may be in powder form for constitution with a suitable vehicle, e.g. sterile pyrogen-free water, before use.

The pharmaceutical composition may be formulated, for example, for immediate release, sustained release, pulsed release, two or more step release, or depot or any other kind of release.

The manufacture of the pharmaceutical compositions according to the present subject matter may be performed according to methods known in the art and will be explained in further detail below. Commonly known and used pharmaceutically acceptable auxiliaries as well as further suitable diluents, flavourings, sweetening agents, colouring agents etc. may be used, depending on the intended mode of administration as well as particular characteristics of the active compound to be used, such as solubility, bioavailability etc.

It is understood, however, that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; and the form of administration. One of ordinary skill in the art would appreciate the variability of such factors and would be able to establish specific dose levels using no more than routine experimentation.

The dosage of exWAGO protein, fragment or nucleic acid encoding a protein or fragment will depend on the age and condition of the patient and the precise dosage will be ultimately at the discretion of the attendant physician or veterinarian. The dosage will also depend on the route of administration.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

The pharmaceutical compositions of the present invention may be given in a single dose or multiple doses.

In one embodiment, the composition of the present invention is administered orally as an initial dose, then one or more booster doses may be provided at a later date. Suitably, the period between the initial dose and each subsequent dose may be 1-2 months.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time are well known in the art.

The optimal formulations can be determined by one skilled in the art depending upon considerations such as the particular ingredients and the desired dosage. See, for example, Remington's Pharmaceutical Sciences, 18th ed. (1990, Mack Publishing Co., Easton, Pa. 18042), pp. 1435-1712, and “Harry's Cosmeticology”, 8th ed. (2000, Chemical Publishing Co., Inc., New York, N.Y. 10016). Such formulations may influence the physical state, stability, rate of in vivo release, and rate of in vivo clearance.

### BRIEF DESCRIPTION OF THE FIGURES

Embodiments of the present invention will now be described, by way of example only, with reference to the accompanying figures in which:

FIG. 1: The exWAGO protein sequence from H. bakeri.

FIG. 2: Closest sequence identity to exWAGO protein sequence from H. bakeri in human genome—alignment to piwi domain of human Argonaute 1 protein.

FIG. 3: Comparative protein sequence alignment of H. bakeri exWAGO protein with the orthologue of N. americanus (SEQ ID NO: 2) showing a high level of identity.

FIG. 4: Comparative sequence alignment of H. bakeri exWAGO protein with the orthologue of T. circumcinta (SEQ ID NO: 3) showing a high level of identity.

FIG. 5: Western blot from an immunoprecipitation of exWAGO from excretory-secretory material of the H. bakeri parasite showing very strong signal in EV-depleted HES (A) (meaning a large portion is not in classically purified extracellular vesicles and is accessible to capture/binding by antibodies) and a lower signal in EVs (B) (10× more protein used for EV-depleted than HES IP). The exWAGO protein is immunopurified from the HES using a polyclonal antibody that only isolates this (and not other) Argonaute proteins, which is confirmed by mass spectrometry analysis of the IP. The antibody is conjugated to Protein L magnetic beads and the complex eluted in SDS/loading buffer prior to being run on a SDS PAGE gel and probed for presence of exWAGO using another antibody that recognizes the denatured form.

FIG. 6: Vaccination strategy using recombinant H. bakeri exWAGO protein in mice and subsequent challenge with H. bakeri, in comparison to HES which is all H. bakeri Excretory-Secretory products. Mice are primed with recombinant exWAGO protein or HES or PBS with Alum at day 0 and then boosted 28 and 35 days later prior to infection on day 42. The mice are sacrificed at 14 and 28 days post infection to assess larval and worm burden, faecal egg count and antibody and cellular response.

FIG. 7: Vaccination results following the strategy of FIG. 4; worm counts per gut section were counted following inoculation and subsequent challenge with H. bakeri at day 14. Total worm counts in the intestine are shown on the left. The normal host niche for H. bakeri is the top section of the intestine (duodenum) and worms in the process of expulsion may be more localized in the bottom section therefore all were counted. The results reveal a significant reduction in adult worm burden at day 28 post infection.

FIG. 8: Vaccination results following the strategy of FIG. 4; egg counts from stool samples following inoculation and subsequent challenge with H. bakeri. Total egg counts in the intestine are shown on the left. The results reveal a significant reduction in faecal egg counts at day 28 post infection.

FIG. 9: Western blot to demonstrate the recognition of post-vaccination, post-worm challenge mouse sera of exWAGO protein which demonstrates that the antibodies against this protein are present in the sera.

FIG. 10: Measurement of IgG1 antibody response to exWAGO at 28 days post infection, n=5 mice per group by ELISA. The recombinant exWAGO protein is used to coat plates so we can measure IgG1 antibodies specific to exWAGO, and then sera from each mouse per group is screened at different dilutions as noted. A secondary IgG antibody is used for detection.

FIG. 11: Comparative expression of various Argonaute proteins in different nematode parasites. Relative expression levels of Argonautes from RNAseq data of the adult free-living parasitic nematode worms. Data are based on the sum of tpm reads of RNAseq data for each orthogroup, normalized to tpm for OG1273 orthogroup (ALG-1/2). The total number of distinct transcripts in each orthogroup in each species is noted below each column. The known Caenorhabditis elegans Argonaute names are used where applicable, or exWAGO. This shows that exWAGOs are the most abundantly expressed of all Argonautes (Supplementary Table S3) in the sheep parasites H. contortus and Teladorsagia circumcincta and the lungworm Angiostrongylus cantonensis.

FIG. 12: Illustrates that exWAGO exists in T. circumcinta in an immune system accessible form (can be captured from the Excretory-secretory products by antibody).

FIG. 13: Provides details of exWAGO orthologues and their sequence identity to SEQ ID NO:1.

FIG. 14: Provides illustrative sequences of exWAGO proteins of noted in FIG. 13.

FIG. 15: indicates protection in H. bakeri model observed at day 28 post infection when using adjuvant Alum.

FIG. 16 indicated exWAGO-specific IgG detected upon infection of sheep with T. circumcinta.

FIG. 17: Provides sequence comparison of H. Corntorus sequence (SEQ ID NO: 8).

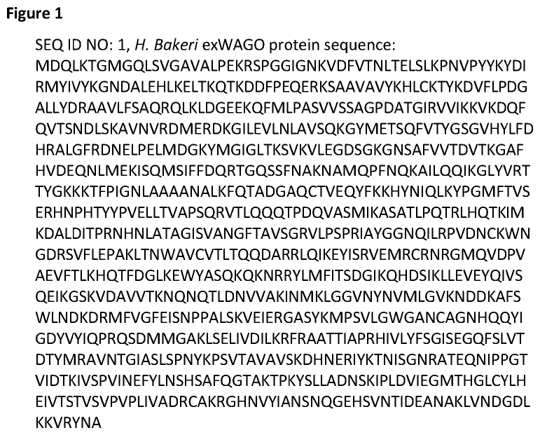

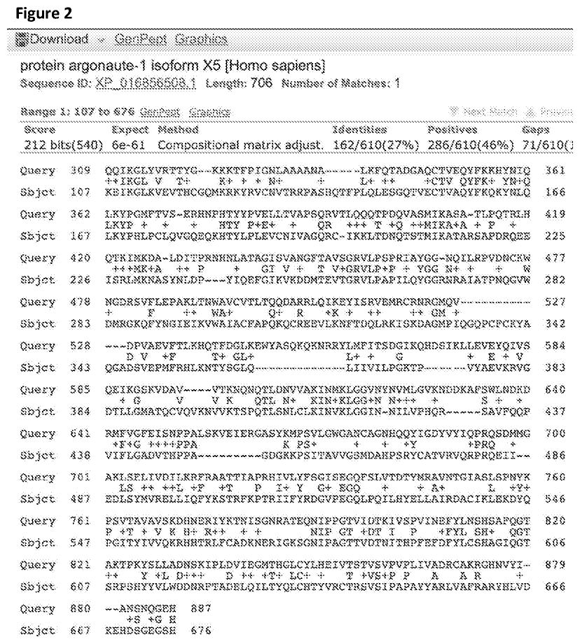

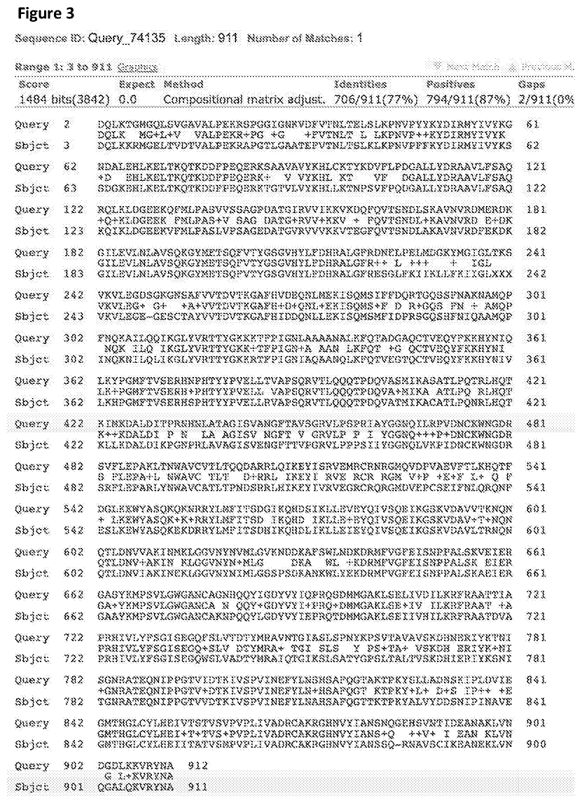

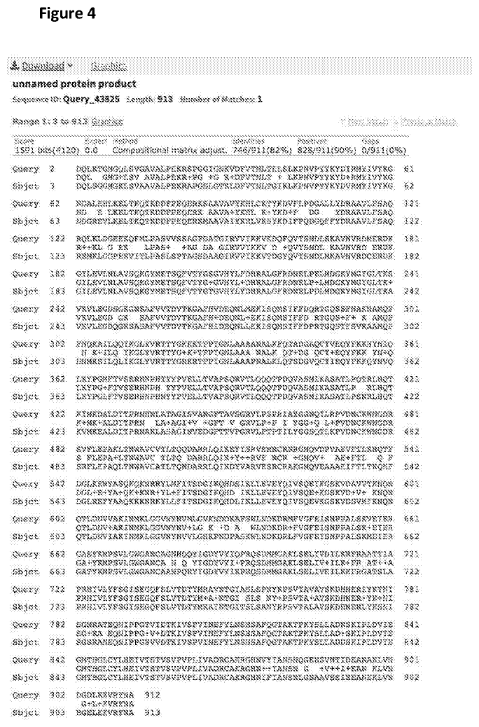

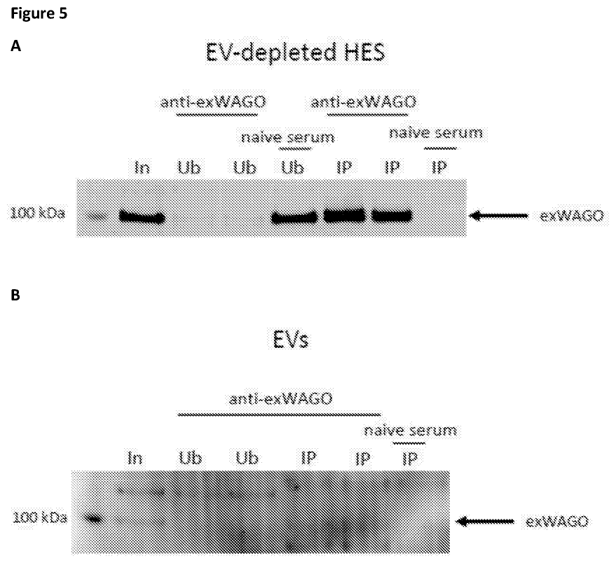

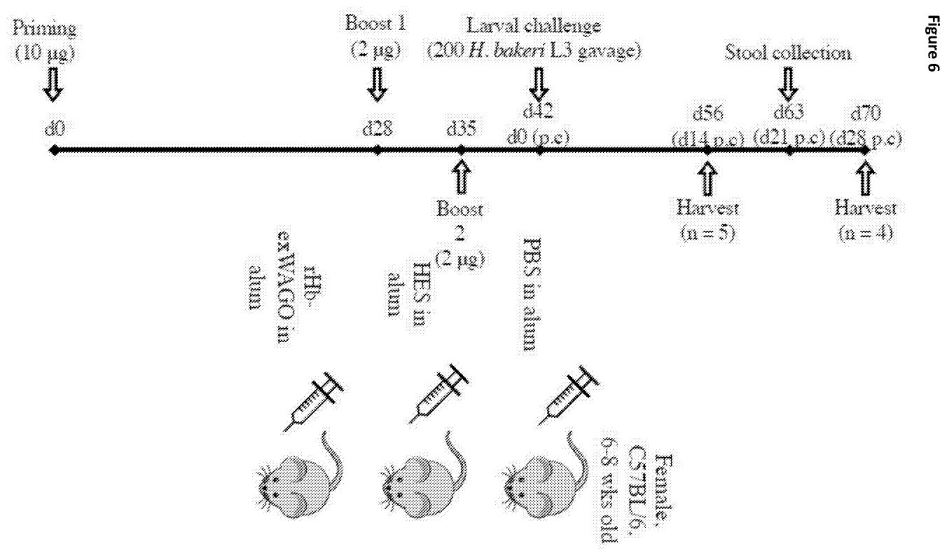

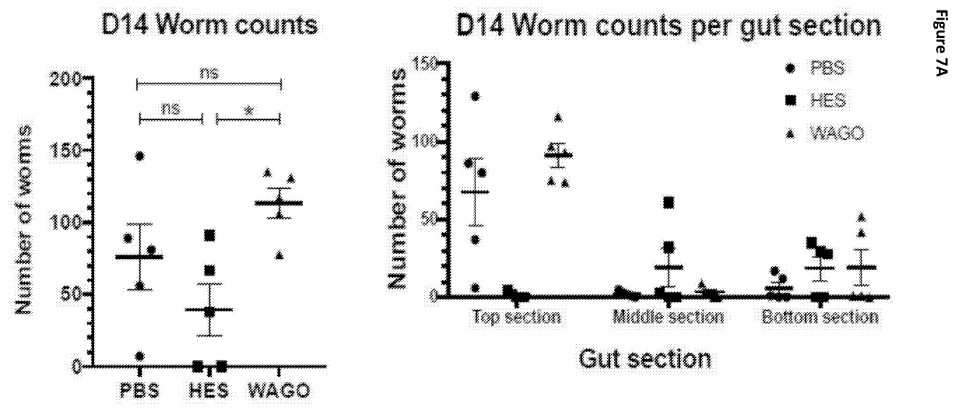

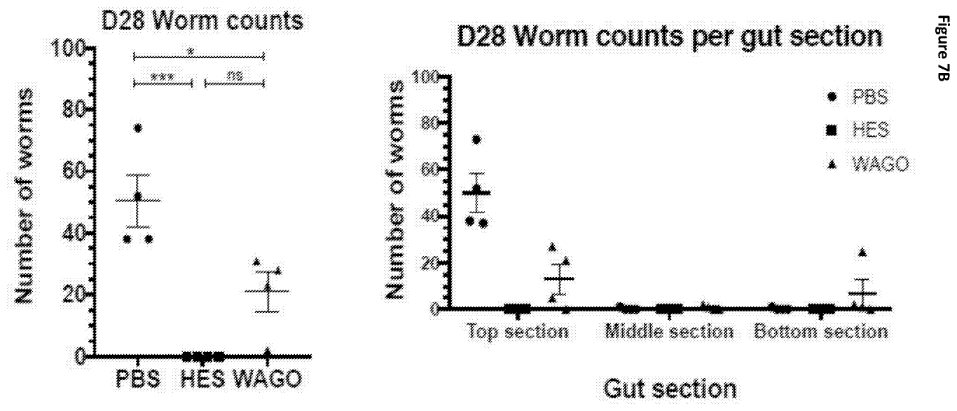

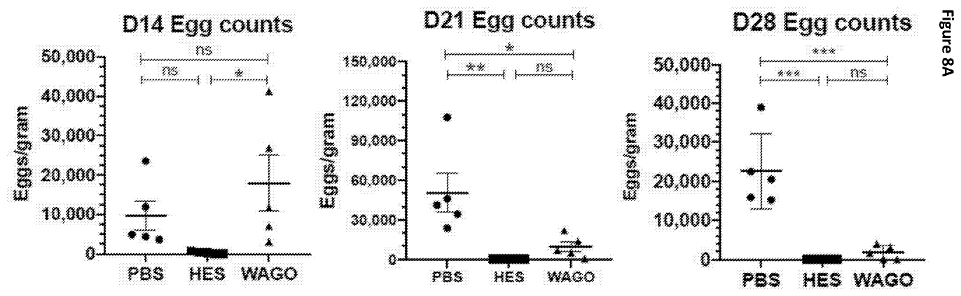

Definitions

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

As used herein, the articles “a” and “an” refer to one or to more than one (for example to at least one) of the grammatical object of the article.

“About” shall generally mean an acceptable degree of error for the quantity measured given the nature or precision of the measurements.

As used herein, the terms “treatment,” “treating,” and the like, refer to obtaining a desired pharmacologic or physiologic effect. The effect may be prophylactic in terms of completely or partially preventing a condition or disease or disorder or symptom thereof and/or may be therapeutic in terms of a partial or complete cure for a condition or disease or disorder and/or adverse symptom or effect attributable to the condition or disease or disorder. For either prophylaxis (prevention) or to cure or reduce the extent of or likelihood of occurrence of the infirmity or malady or condition or event in the instance where the patient is afflicted.

“Treatment” for example, covers any treatment of a condition or disease in a mammal, particularly in a human, and includes: (a) preventing the condition or disease, disorder or symptom thereof from occurring in a subject which may be predisposed to the condition or disease or disorder but has not yet been diagnosed as having it; (b) inhibiting the condition or disease, disorder or symptom thereof, such as, arresting its development; and (c) relieving, alleviating or ameliorating the condition or disease or disorder or symptom thereof, such as, for example, causing regression of the condition or disease or disorder or symptom thereof. As used herein, the term “effective amount” means that amount of an agent, for example protein that will elicit the biological or medical response of a tissue, system, animal or human that is being sought, for instance, by a researcher, clinician or veterinarian.

As used herein, “pharmaceutically acceptable excipient” or “pharmaceutically acceptable carrier” mean a pharmaceutically acceptable material, composition or vehicle involved in giving form or consistency to the composition for example the protein and adjuvant. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be pharmaceutically-acceptable e.g. of sufficiently high purity.

The term “combination” as used herein refers to either a fixed combination in one dosage unit form, or non-fixed combination. The term “fixed combination” means that the active ingredients, e.g. a protein and a combination partner, for example an antihelmintic or another antigen are both administered to a patient simultaneously in the form of a single entity or dosage.

The term “non-fixed combination” means that the active ingredients, e.g. an exWAGO protein (1) and a combination partner, (e.g. another drug or antigen as explained below, also referred to as “therapeutic agent” or “co-agent”) are both administered to a patient as separate entities either simultaneously, concurrently or sequentially with no, specific time limits, wherein such administration provides therapeutically effective levels of the two compounds in the body of the patient.

Herein, “Heligmosomoides polygyrus” and “Heligmosomoides bakeri” are used interchangeably.

As used herein, a peptide for example a fragment of exWAGO protein can include any polymer consisting of at least two amino acids which are covalently linked to each other, preferably via a peptide bond. Preferably a peptide consists of two to ten amino acids. Suitably, the peptide can be an oligopeptide which comprises from 10 to 100 amino acids, suitably covalently linked to each other, preferably via a peptide bond. Suitably, the peptide can be a protein comprising a plurality of amino acids which are covalently linked to each other and which comprise at least 100 amino acids. Suitably a protein may also be two or more peptides each comprising of at least two amino acids, non-covalently linked to each other.

### DETAILED DESCRIPTION OF THE INVENTION

Embodiments of the present invention will now be described by way of example only with reference to the accompanying figures.

### EXAMPLE 1

### Screening H. bakeri Excretory-Secretory Material (HES) for exWAGO in the Presence and Absence of EVs

ExWAGO protein was captured from excretory-secretory products using immunopurification with exWAGO antibodies attached to beads. The ES products that are screened were either vesicles (where the ES is first ultracentrifuged and the pellet is used) or non-vesicular material that is the supernatant of the ultracentrifugation. The Immunoprecipitate products are then eluted from the beads and run on a gel.

### EXAMPLE 2

### Protective Role of Inoculation by exWAGO Against Future Parasitic Infection

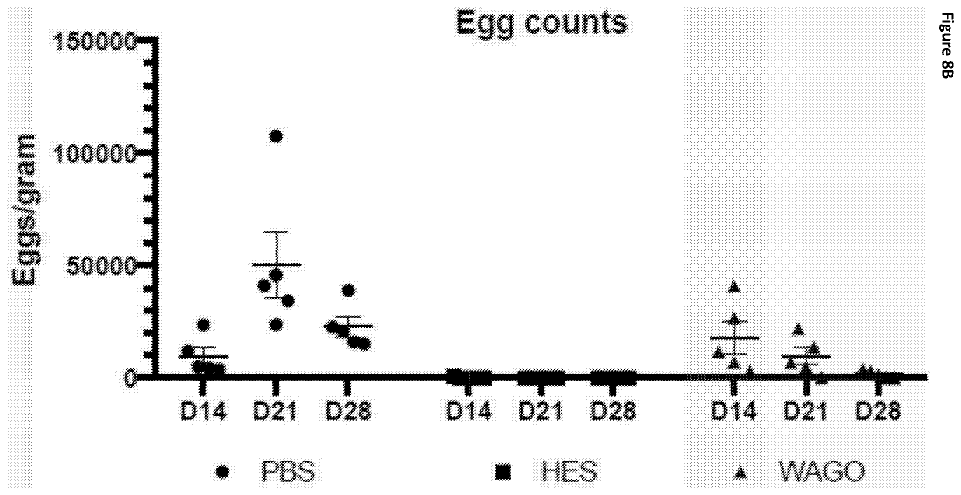

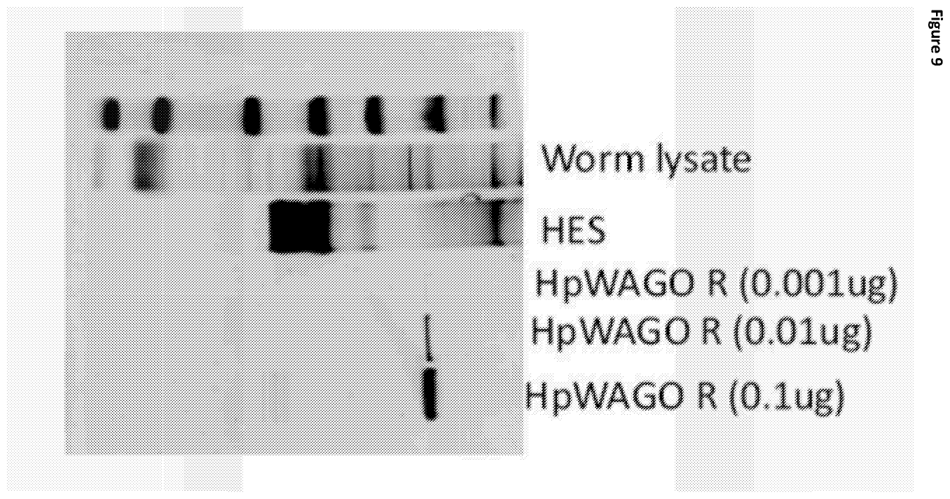

A vaccination protocol was proposed (as shown in FIG. 6), using a vehicle only control of PBS and alum, a positive control of H. bakeri HES and alum, and recombinant H. bakeri exWAGO and alum. On day 0, 10 female C57BL/6 mice aged 6-8 weeks per condition were inoculated via intraperitoneal (IP) injection with 10 μg of each condition. At days 28 and 35, booster IP inoculations of 2 μg were provided to the mice.

On day 42 (day 0 of post-challenge, p.c.) the mice were each provided with 200 H. bakeri larval infective (L3) nematode worms by gavage.

Samples were collected and measurements taken of:
1) Worm counts (d56 [d14 p.c.] (n=5), d70 [d28 p.c.] (n=4)).
2) Egg counts (d63 [d21 p.c.] (n=5))
3) Serum
4) Extra stools
5) Tissue and gut sections (d56 [d14 p.c.] (n=5), d70 [d28 p.c.] (n=4)).

Vaccination with Recombinant exWAGO Results in Protection against Subsequent Infection at 28 Days Post Infection

Following the above inoculation protocol, gut sections of mice were sampled at day d56 [d14 p.c.] (n=5) and at day d70 [d28 p.c.] (n=4), and screened for the number of adult H. bakeri worms was counted.

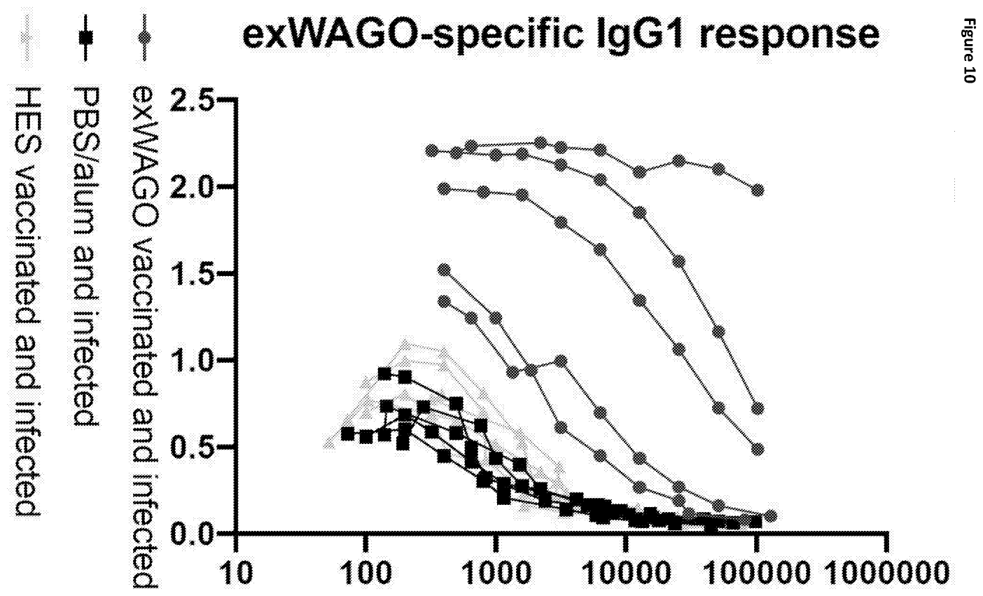

As shown in FIG. 7, vaccination with recombinant H. bakeri exWAGO results in protection from subsequent infection at 28 days post infection. This shows a 58% reduction in adult worm counts. Those found only in the bottom section are presumably in the process of exiting the gut. FIG. 7A: 14 days post-infection,

FIG. 7B: 28 days post-infection.

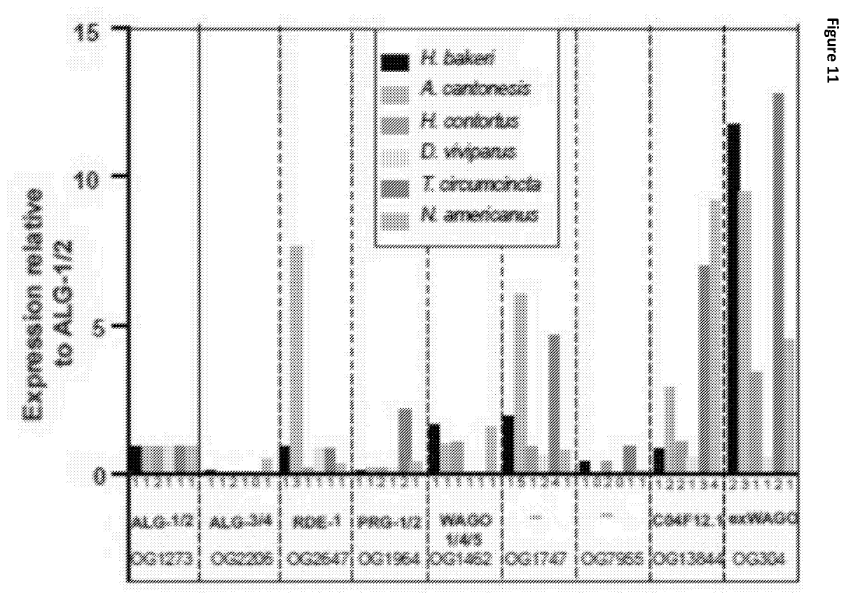

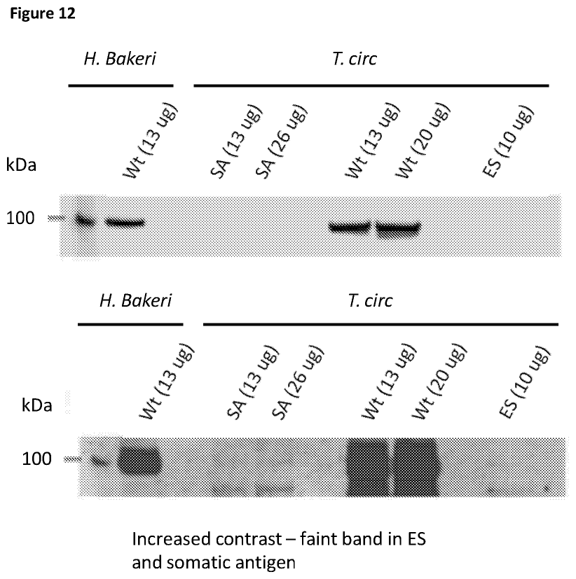

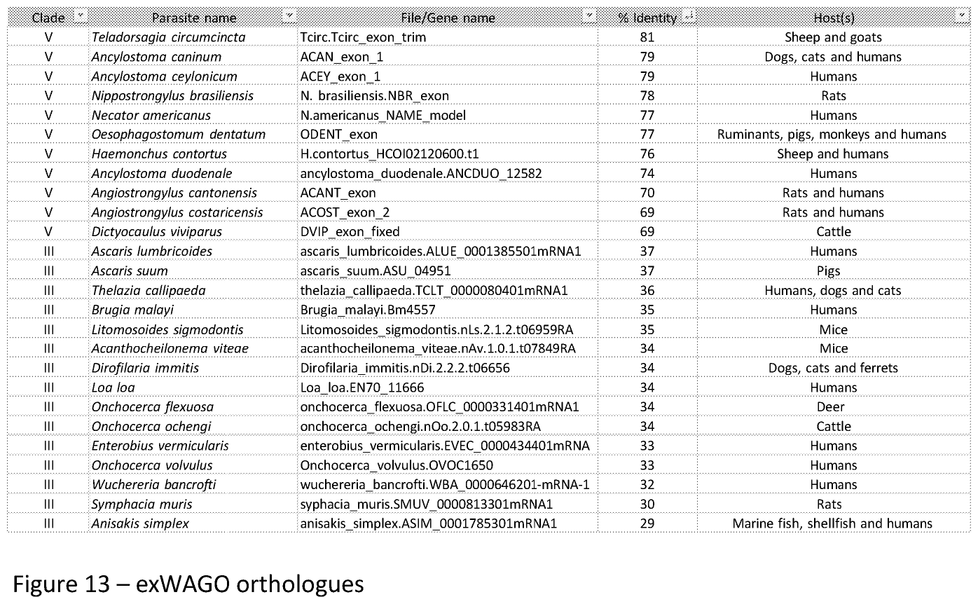

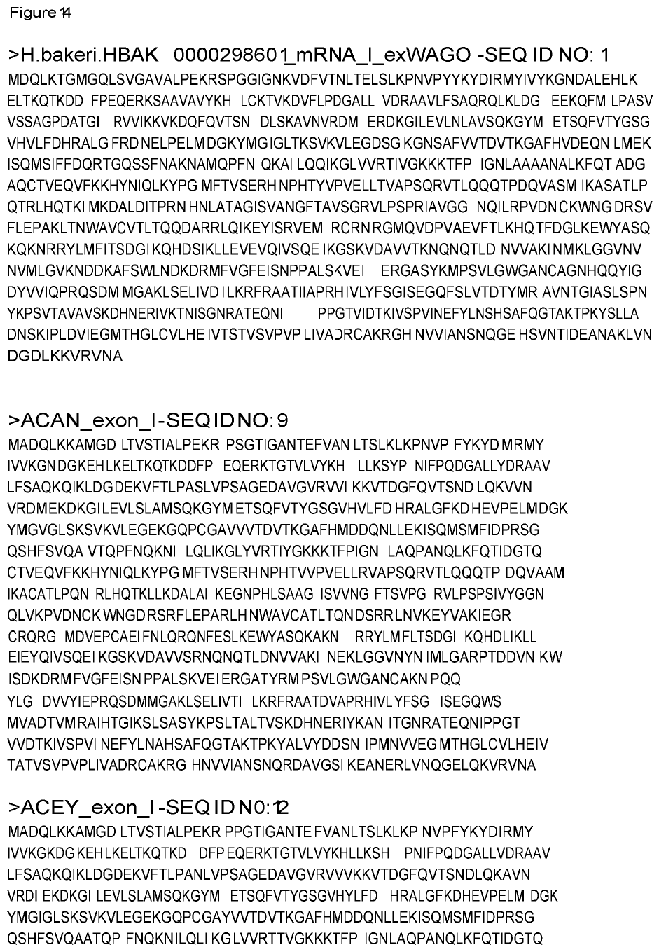

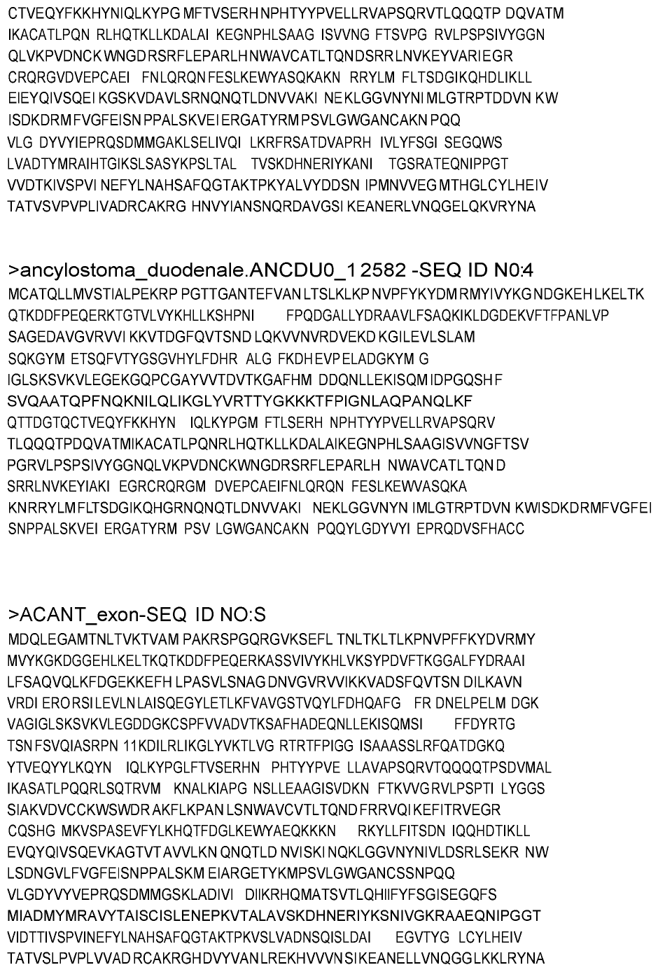

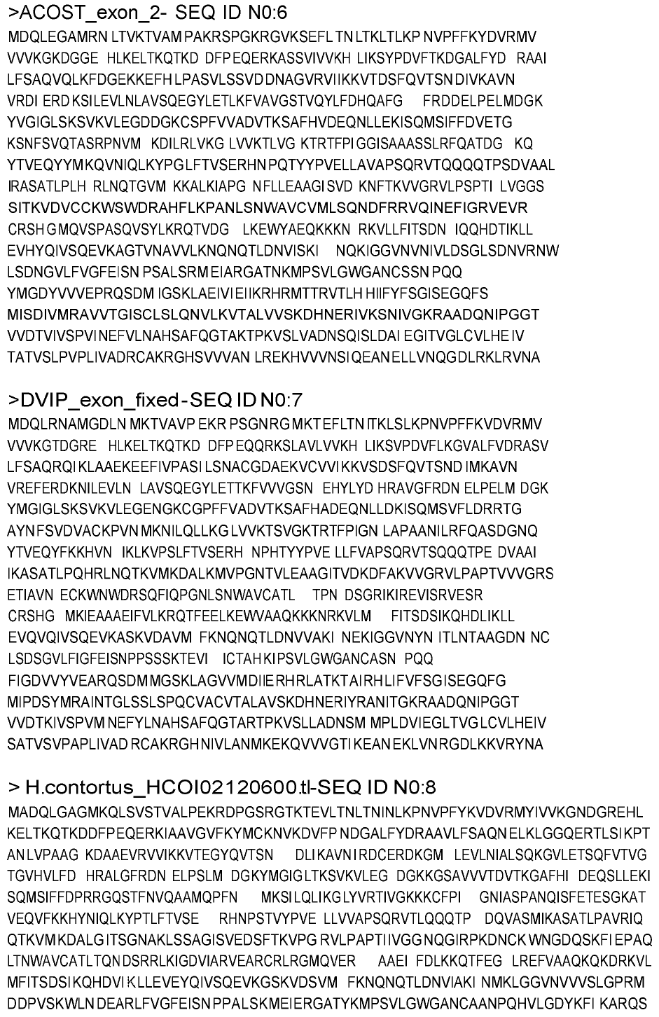

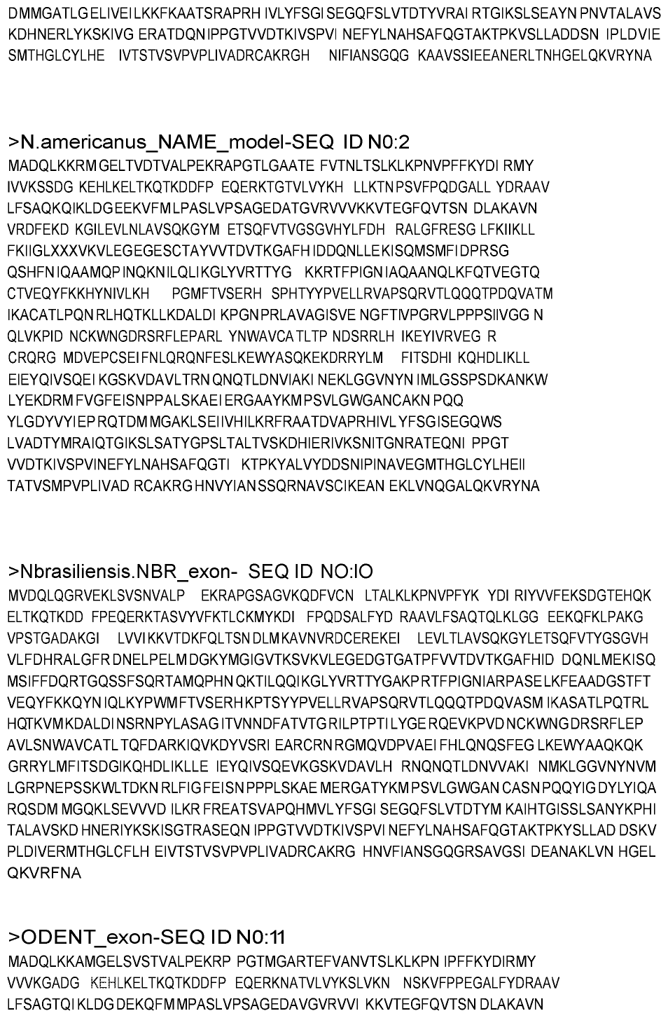

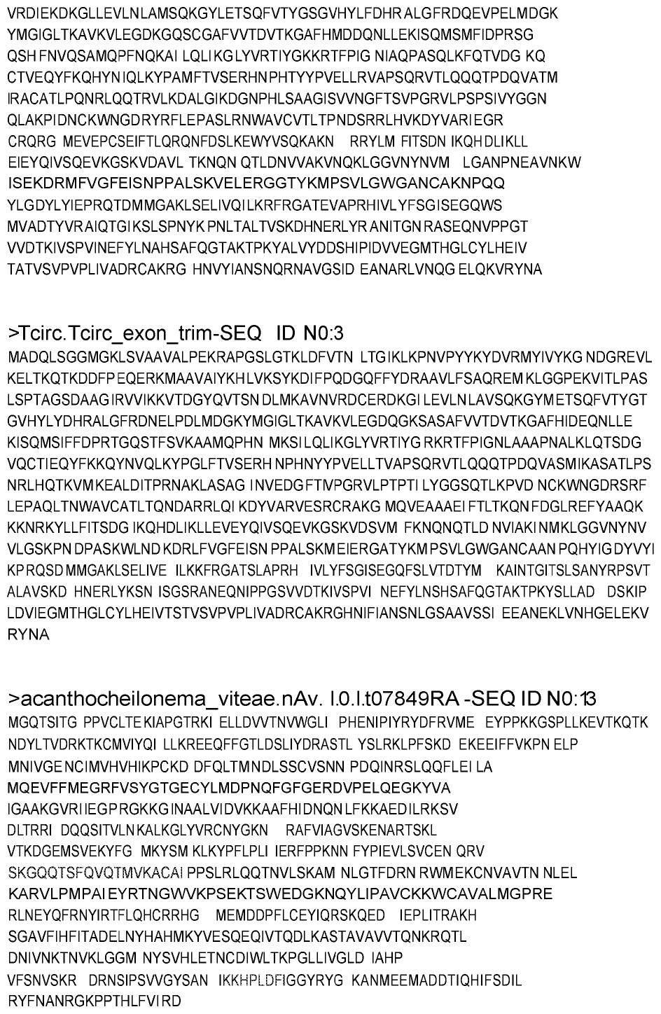

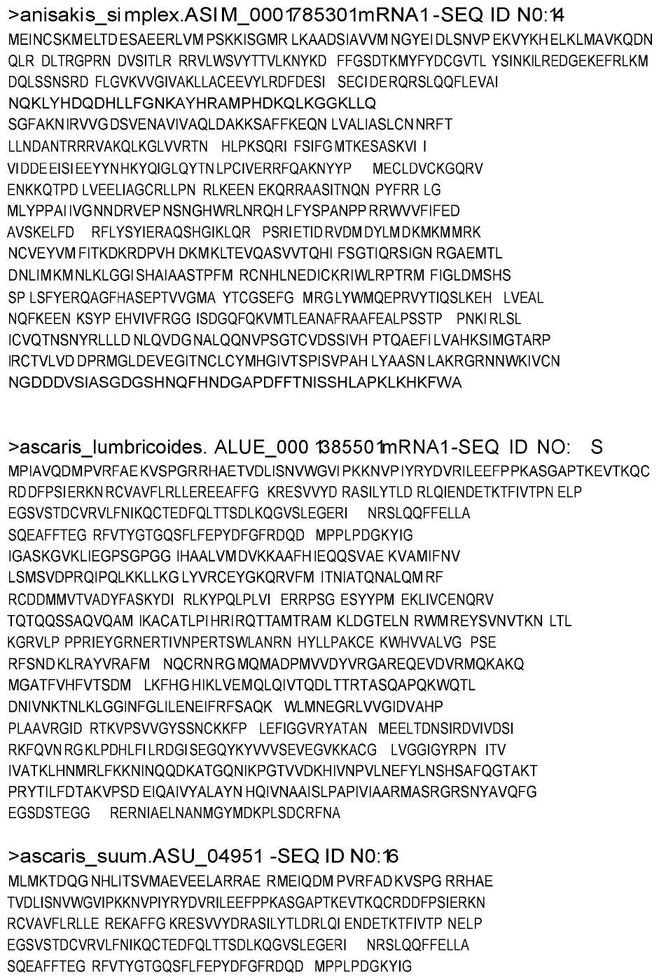

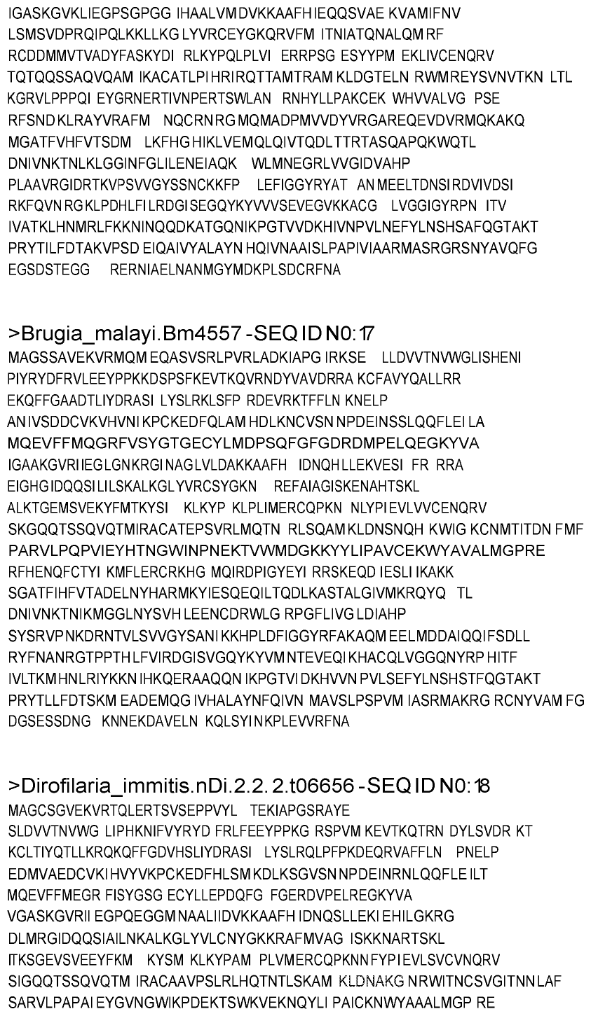

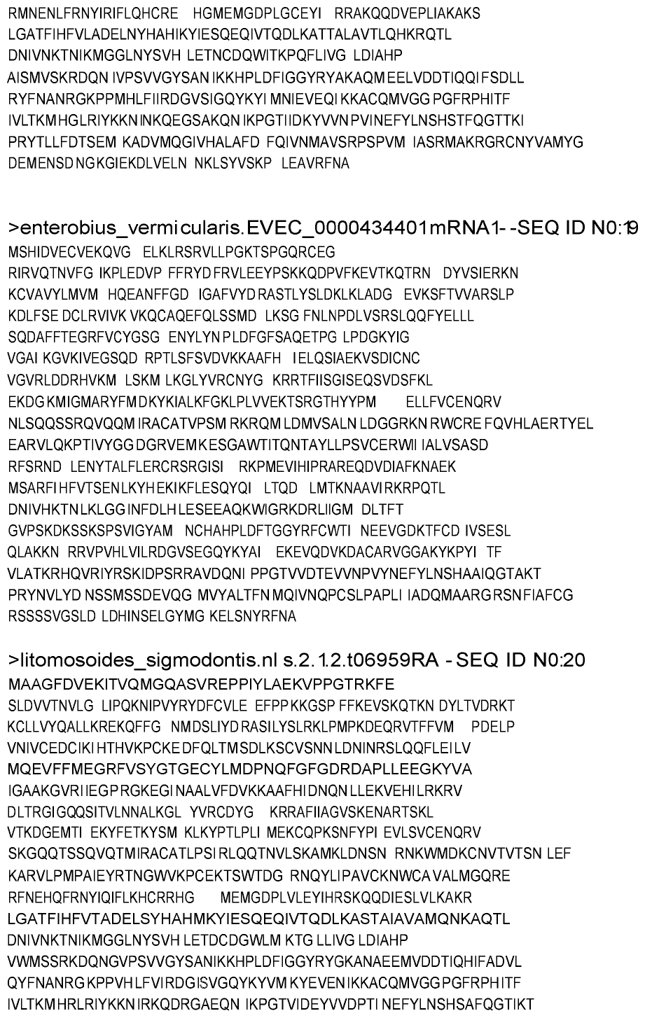

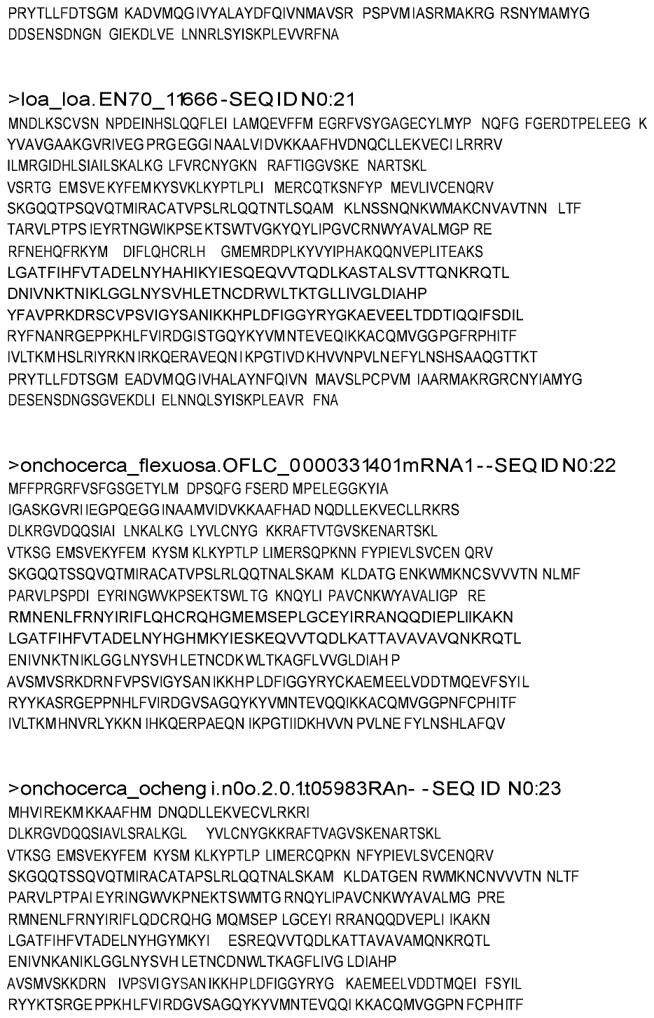

As shown in FIG. 8, stool samples were taken at days 14, 21 and 28 and the number of H. bakeri eggs were then counted. This clearly demonstrates that inoculation with recombinant H. bakeri exWAGO results in protection from subsequent infection with a 92% (average) reduction in egg counts in the exWAGO experiment. FIG. 8A: Change in egg count at 14, 21 and 29 days, FIG. 8B: collated egg count/day data.

As shown in FIG. 9, antibodies generated during vaccination recognise exWAGO: Western blot of 1 μg worm lysate, 1 μg total excretory-secretory material (HES) or the noted concentrations of the recombinant exWAGO protein, incubated with the sera of mice vaccinated with exWAGO and infected.

As shown in FIG. 10, mice which had been vaccinated with recombinant exWAGO demonstrated a significantly higher IgG1 antibody response to exWAGO (by ELISA, coated with recombinant exWAGO) at 28 days post infection, n=5 mice per group.

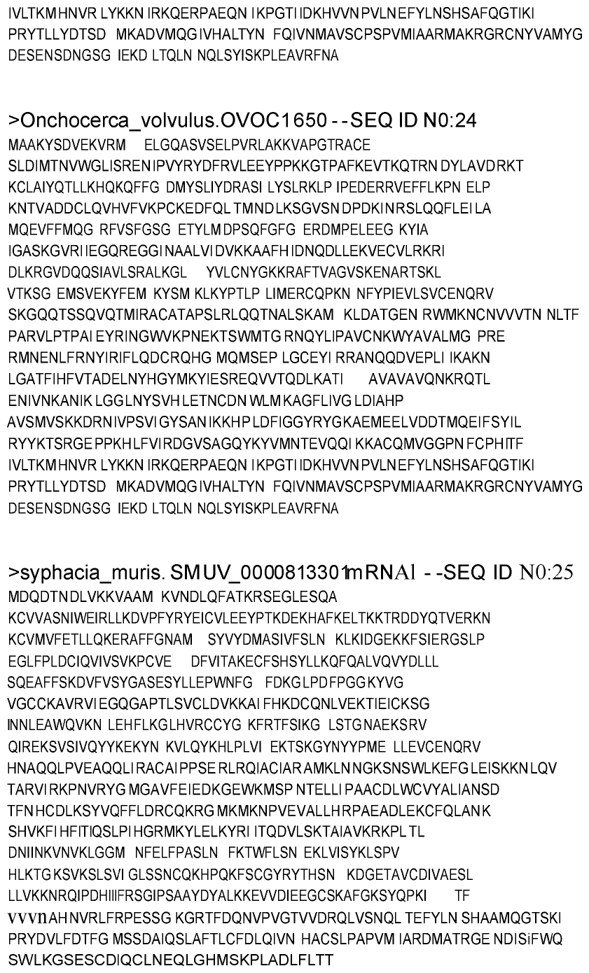

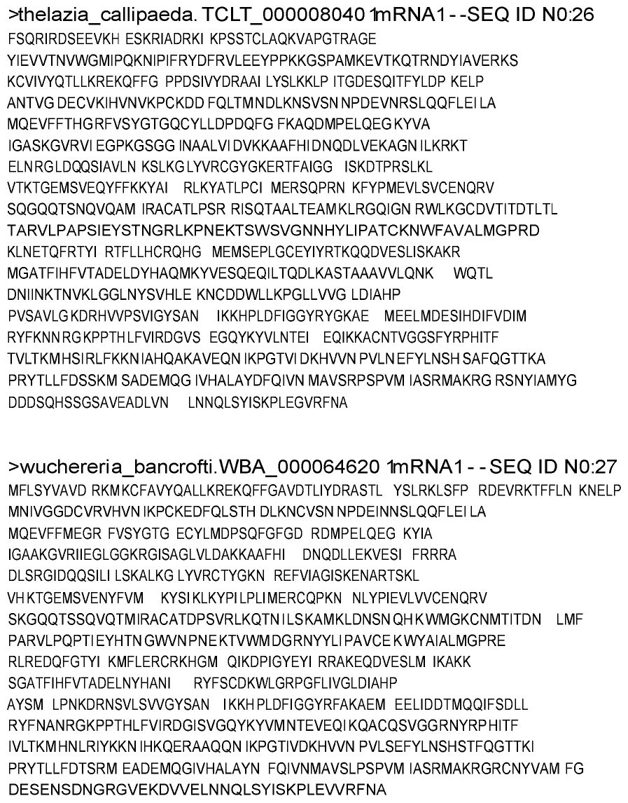

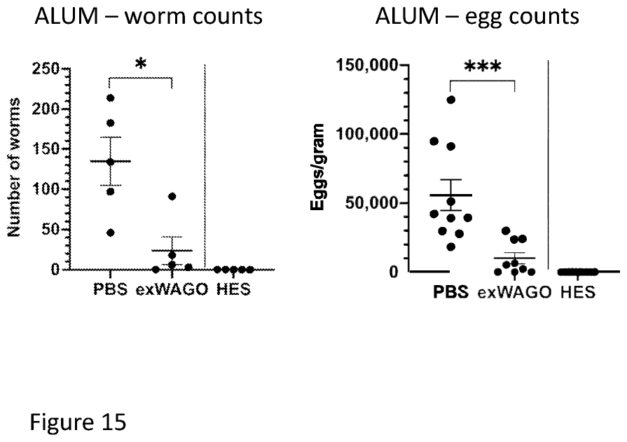

As shown in FIG. 15, using Alum as an adjuvant, exWAGO provides protection in the model.

### EXAMPLE 3

### Comparative Expression of Various Argonaute Proteins in Different Nematode Parasites

As shown in FIG. 11, relative expression levels of different classes of Argonautes from adult free-living parasitic nematode worms were compared, using RNAseq data. Data was based on the sum of tpm reads of RNAseq data for each orthogroup, normalized to tpm for 0G1273 orthogroup (ALG-1/2). The total number of distinct transcripts in each orthogroup in each species is noted below each column. The known Caenorhabditis elegans Argonaute names are used where applicable, or exWAGO. This shows that exWAGOs are the most abundantly expressed of all Argonautes in the sheep parasites H. contortus and Teladorsagia circumcincta and the lungworm Angiostrongylus cantonensis.

### EXAMPLE 4

### exWAGO Specific IgG Detected upon Infection of Sheep with T. circumcinta

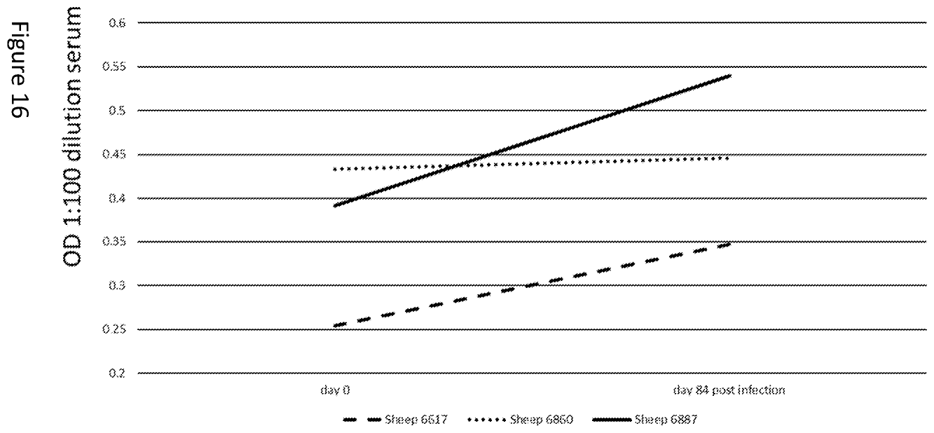

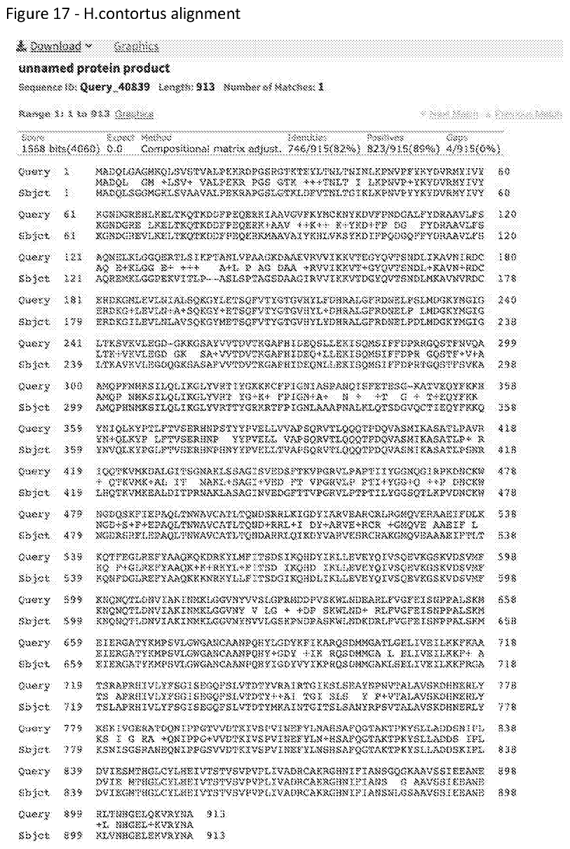

As shown in FIG. 16, using a sheep model instead of a mouse model with exWAGO from the parasite T. circumcinta, it was considered that IgG levels were modulated in the host following treatment. Thus, it is considered that the protective effect of exWAGO as illustrated in the H. bakeri model will be observed in other animals.

Preferred compositions, features and embodiments of each aspect of the invention are as for each of the other aspects mutatis mutandis unless context demands otherwise.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

Although the invention has been particularly shown and described with reference to particular examples, it will be understood by those skilled in the art that various changes in the form and details may be made therein without departing from the scope of the present invention.

1. An exWAGO protein or fragment thereof from a parasitic nematode or a nucleic acid sequence capable of expressing exWAGO or a fragment thereof for use in raising a protective immune response in an animal.

2. An exWAGO protein for use in raising a protective immune response in an animal as claimed in claim 1 wherein the exWAGO protein comprises a sequence of SEQ ID NO: 1 or has a sequence identity of at least 60% to a portion of SEQ ID NO: 1.

3. An exWAGO protein for use in raising a protective immune response in an animal as claimed in claim 1 wherein the exWAGO protein comprises a sequence of SEQ ID NO: 1 or has a sequence identity of at least 60% to SEQ ID NO: 1.

4. A composition comprising an exWAGO protein or a fragment thereof or a nucleic acid sequence capable of expressing exWAGO comprising a sequence of SEQ ID NO: 1 or with a sequence identity of at least 60% to SEQ ID NO: 1 and optionally an adjuvant for use in the treatment or prophylaxis of a parasitic nematode.

5. A composition as claimed in claim 4 further comprising at least a second parasitic antigen to which an immune response may be raised.

6. An exWAGO protein for use in raising an immune response in an animal as claimed in claim 1 wherein the parasitic nematode is selected from the group comprising: parasitic nematodes of Clade V and Clade III, optionally wherein Clade V includes:
Strongylida
Rhabditida
Diplogasterida
and
Clade III includes:
Oxyurida
Spirurida
Rhigonematida
Ascaridida.

7. An exWAGO protein for use in raising an immune response in an animal as claimed in claim 1 wherein the parasitic nematode is selected from the group comprising
Nippostrongylus brasiliensis (rodents)
Ancylostoma caninum
Ancylostoma ceylanicum
Ancylostoma duodenale
Oesophagostomum dentatum
Angiostrongylus costaricensis
Dictyocaulus viviparous
Symphacia muris (rodents)
Enterobius vermicularis
Anisakis simplex
Ascaris suum
Ascaris lumbricoides
Thelazia callipaeda
Brugia malayi
Wuchereria bancrofti
Onchocerca flexuosa
Onchocerca ochengi, and
Acanthocheilonema viteae (rodents).

8. An exWAGO protein for use in raising an immune response in an animal as claimed in claim 1 wherein the parasitic nematode is selected from the group comprising Heligmosomoides polygyrus, Heligmosomoides bakeri, Teladorsagia circumcincta, Ancylostoma caninum, Ancylostoma ceylonicum, Nippostrongylus brasiliensis, Necator americanus, Oesophagostomum dentatum, Haemonchus contortus, Ancylostoma duodenale, Angiostrongylus cantonensis, Angiostrongylus costaricensis, Nematodirus, Ostertagia ostertagi, and Dictyocaulus viviparous.

9. An exWAGO protein for use in raising an immune response in an animal as claimed in claim 1 wherein the parasitic nematode is selected from the group comprising Ascaris lumbricoides, Ascaris suum, Thelazia callipaeda, Brugia malayi, Litomosoides sigmodontis, Acanthocheilonema viteae, Dirofilaria immitis, Loa loa, Onchocerca flexuosa, Onchocerca ochengi, Enterobius vermicularis, Onchocerca volvulus, Wuchereria bancrofti, and Symphacia muris.

10. An exWAGO protein for use in raising an immune response in an animal as claimed in claim 1 wherein an immune response is against a parasitic nematode of a different clade of parasitic nematode to that of Heligmosomoides polygyrus.

11. An exWAGO protein for use in raising an immune response in an animal as claimed in claim 1 wherein an immune response is against a parasitic nematode of the same clade of parasitic nematode to that of Heligmosomoides polygyrus.

12. An exWAGO protein for use in raising an immune response in an animal as claimed in claim 1 wherein the animal is selected from human, cat, dog, camelid, equid, equine, or ovine or bovine.

13. A method of raising an immune response against a parasitic nematode in an animal said method comprising the step of administering to the animal exWAGO protein or fragment thereof or a nucleic acid sequence capable of expressing exWAGO or a fragment thereof sufficient to induce an immune response against the parasitic nematode.

14. A method of raising an immune response against a parasitic nematode in an animal as claimed in claim 13 said method comprising the step of administering to the animal a composition comprising an exWAGO protein or a fragment thereof or a nucleic acid sequence capable of expressing exWAGO comprising a sequence of SEQ ID NO: 1 or with a sequence identity of at least 60% to SEQ ID NO: 1.

15. A method to determine an epitope of exWAGO to which antibodies or aptamers can be raised wherein the antibodies or aptamers provide a protective immune response to a host subsequently infected with a parasitic nematode the method comprising the steps of determining an antibody or antibodies that associate with exWAGO under native in vivo conditions, determining with the antibody or antibodies that block the ability of exWAGO to interact with host or microbial genes in vivo.


SEQUENCE LISTING (27 sequences)

SEQ ID NO: 1 - protein, 912 aa, Heligmosomoides bakeri
MDQLKTGMGQLSVGAVALPEKRSPGGIGNKVDFVTNLTELSLKPNVPYYKYDIRMYIVYKGNDALEHLKELTKQTKDDFPEQERKSAAVAVYKHLCKTYKDVFLPDGALLYDRAAVLFSAQRQLKLDGEEKQFMLPASVVSSAGPDATGIRVVIKKVKDQFQVTSNDLSKAVNVRDMERDKGILEVLNLAVSQKGYMETSQFVTYGSGVHYLFDHRALGFRDNELPELMDGKYMGIGLTKSVKVLEGDSGKGNSAFVVTDVTKGAFHVDEQNLMEKISQMSIFFDQRTGQSSFNAKNAMQPFNQKAILQQIKGLYVRTTYGKKKTFPIGNLAAAANALKFQTADGAQCTVEQYFKKHYNIQLKYPGMFTVSERHNPHTYYPVELLTVAPSQRVTLQQQTPDQVASMIKASATLPQTRLHQTKIMKDALDITPRNHNLATAGISVANGFTAVSGRVLPSPRIAYGGNQILRPVDNCKWNGDRSVFLEPAKLTNWAVCVTLTQQDARRLQIKEYISRVEMRCRNRGMQVDPVAEVFTLKHQTFDGLKEWYASQKQKNRRYLMFITSDGIKQHDSIKLLEVEYQIVSQEIKGSKVDAVVTKNQNQTLDNVVAKINMKLGGVNYNVMLGVKNDDKAFSWLNDKDRMFVGFEISNPPALSKVEIERGASYKMPSVLGWGANCAGNHQQYIGDYVYIQPRQSDMMGAKLSELIVDILKRFRAATTIAPRHIVLYFSGISEGQFSLVTDTYMRAVNTGIASLSPNYKPSVTAVAVSKDHNERIYKTNISGNRATEQNIPPGTVIDTKIVSPVINEFYLNSHSAFQGTAKTPKYSLLADNSKIPLDVIEGMTHGLCYLHEIVTSTVSVPVPLIVADRCAKRGHNVYIANSNQGEHSVNTIDEANAKLVNDGDLKKVRYNA

SEQ ID NO: 2 - protein, 911 aa, Necator americanus
  misc_feature (240)..(242): Xaa can be any naturally occurring amino acid
MADQLKKRMGELTVDTVALPEKRAPGTLGAATEFVTNLTSLKLKPNVPFFKYDIRMYIVYKSSDGKEHLKELTKQTKDDFPEQERKTGTVLVYKHLLKTNPSVFPQDGALLYDRAAVLFSAQKQIKLDGEEKVFMLPASLVPSAGEDATGVRVVVKKVTEGFQVTSNDLAKAVNVRDFEKDKGILEVLNLAVSQKGYMETSQFVTYGSGVHYLFDHRALGFRESGLFKIIKLLFKIIGLXXXVKVLEGEGESCTAYVVTDVTKGAFHIDDQNLLEKISQMSMFIDPRSGQSHFNIQAAMQPINQKNILQLIKGLYVRTTYGKKRTFPIGNIAQAANQLKFQTVEGTQCTVEQYFKKHYNIVLKHPGMFTVSERHSPHTYYPVELLRVAPSQRVTLQQQTPDQVATMIKACATLPQNRLHQTKLLKDALDIKPGNPRLAVAGISVENGFTTVPGRVLPPPSIIYGGNQLVKPIDNCKWNGDRSRFLEPARLYNWAVCATLTPNDSRRLHIKEYIVRVEGRCRQRGMDVEPCSEIFNLQRQNFESLKEWYASQKEKDRRYLMFITSDHIKQHDLIKLLEIEYQIVSQEIKGSKVDAVLTRNQNQTLDNVIAKINEKLGGVNYNIMLGSSPSDKANKWLYEKDRMFVGFEISNPPALSKAEIERGAAYKMPSVLGWGANCAKNPQQYLGDYVYIEPRQTDMMGAKLSEIIVHILKRFRAATDVAPRHIVLYFSGISEGQWSLVADTYMRAIQTGIKSLSATYGPSLTALTVSKDHIERIYKSNITGNRATEQNIPPGTVVDTKIVSPVINEFYLNAHSAFQGTTKTPKYALVYDDSNIPINAVEGMTHGLCYLHEIITATVSMPVPLIVADRCAKRGHNVYIANSSQRNAVSCIKEANEKLVNQGALQKVRYNA

SEQ ID NO: 3 - protein, 913 aa, Teladorsagia circumcincta
MADQLSGGMGKLSVAAVALPEKRAPGSLGTKLDFVTNLTGIKLKPNVPYYKYDVRMYIVYKGNDGREVLKELTKQTKDDFPEQERKMAAVAIYKHLVKSYKDIFPQDGQFFYDRAAVLFSAQREMKLGGPEKVITLPASLSPTAGSDAAGIRVVIKKVTDGYQVTSNDLMKAVNVRDCERDKGILEVLNLAVSQKGYMETSQFVTYGTGVHYLYDHRALGFRDNELPDLMDGKYMGIGLTKAVKVLEGDQGKSASAFVVTDVTKGAFHIDEQNLLEKISQMSIFFDPRTGQSTFSVKAAMQPHNMKSILQLIKGLYVRTTYGRKRTFPIGNLAAAPNALKLQTSDGVQCTIEQYFKKQYNVQLKYPGLFTVSERHNPHNYYPVELLTVAPSQRVTLQQQTPDQVASMIKASATLPSNRLHQTKVMKEALDITPRNAKLASAGINVEDGFTTVPGRVLPTPTILYGGSQTLKPVDNCKWNGDRSRFLEPAQLTNWAVCATLTQNDARRLQIKDYVARVESRCRAKGMQVEAAAEIFTLTKQNFDGLREFYAAQKKKNRKYLLFITSDGIKQHDLIKLLEVEYQIVSQEVKGSKVDSVMFKNQNQTLDNVIAKINMKLGGVNYNVVLGSKPNDPASKWLNDKDRLFVGFEISNPPALSKMEIERGATYKMPSVLGWGANCAANPQHYIGDYVYIKPRQSDMMGAKLSELIVEILKKFRGATSLAPRHIVLYFSGISEGQFSLVTDTYMKAINTGITSLSANYRPSVTALAVSKDHNERLYKSNISGSRANEQNIPPGSVVDTKIVSPVINEFYLNSHSAFQGTAKTPKYSLLADDSKIPLDVIEGMTHGLCYLHEIVTSTVSVPVPLIVADRCAKRGHNIFIANSNLGSAAVSSIEEANEKLVNHGELEKVRYNA

SEQ ID NO: 4 - protein, 666 aa, Ancylostoma duodenale
MCATQLLMVSTIALPEKRPPGTTGANTEFVANLTSLKLKPNVPFYKYDMRMYIVYKGNDGKEHLKELTKQTKDDFPEQERKTGTVLVYKHLLKSHPNIFPQDGALLYDRAAVLFSAQKIKLDGDEKVFTFPANLVPSAGEDAVGVRVVIKKVTDGFQVTSNDLQKVVNVRDVEKDKGILEVLSLAMSQKGYMETSQFVTYGSGVHYLFDHRALGFKDHEVPELADGKYMGIGLSKSVKVLEGEKGQPCGAYVVTDVTKGAFHMDDQNLLEKISQMIDPGQSHFSVQAATQPFNQKNILQLIKGLYVRTTYGKKKTFPIGNLAQPANQLKFQTTDGTQCTVEQYFKKHYNIQLKYPGMFTLSERHNPHTYYPVELLRVAPSQRVTLQQQTPDQVATMIKACATLPQNRLHQTKLLKDALAIKEGNPHLSAAGISVVNGFTSVPGRVLPSPSIVYGGNQLVKPVDNCKWNGDRSRFLEPARLHNWAVCATLTQNDSRRLNVKEYIAKIEGRCRQRGMDVEPCAEIFNLQRQNFESLKEWYASQKAKNRRYLMFLTSDGIKQHGRNQNQTLDNVVAKINEKLGGVNYNIMLGTRPTDVNKWISDKDRMFVGFEISNPPALSKVEIERGATYRMPSVLGWGANCAKNPQQYLGDYVYIEPRQDVSFHACC

SEQ ID NO: 5 - protein, 909 aa, Angiostrongylus cantonensis
MDQLEGAMTNLTVKTVAMPAKRSPGQRGVKSEFLTNLTKLTLKPNVPFFKYDVRMYMVYKGKDGGEHLKELTKQTKDDFPEQERKASSVIVYKHLVKSYPDVFTKGGALFYDRAAILFSAQVQLKFDGEKKEFHLPASVLSNAGDNVGVRVVIKKVADSFQVTSNDILKAVNVRDIERDRSILEVLNLAISQEGYLETLKFVAYGSTVQYLFDHQAFGFRDNELPELMDGKYAGIGLSKSVKVLEGDDGKCSPFVVADVTKSAFHADEQNLLEKISQMSIFFDYRTGTSNFSVQIASRPNIIKDILRLIKGLYVKTLYGRTRTFPIGGISAAASSLRFQATDGKQYTVEQYYLKQYNIQLKYPGLFTVSERHNPHTYYPVELLAVAPSQRVTQQQQTPSDVMALIKASATLPQQRLSQTRVMKNALKIAPGNSLLEAAGISVDKNFTKVVGRVLPSPTILYGGSSIAKVDVCCKWSWDRAKFLKPANLSNWAVCVTLTQNDFRRVQIKEFITRVEGRCQSHGMKVSPASEVFYLKHQTFDGLKEWYAEQKKKNRKYLLFITSDNIQQHDTIKLLEVQYQIVSQEVKAGTVTAVVLKNQNQTLDNVISKINQKLGGVNYNIVLDSRLSEKRNWLSDNGVLFVGFEISNPPALSKMEIARGETYKMPSVLGWGANCSSNPQQYLGDYVYVEPRQSDMMGSKLADIVIDIIKRHQMATSVTLQHIIFYFSGISEGQFSMIADMYMRAVYTAISCISLENEPKVTALAVSKDHNERIYKSNIVGKRAAEQNIPGGTVIDTTIVSPVINEFYLNAHSAFQGTAKTPKYSLVADNSQISLDAIEGVTYGLCYLHEIVTATVSLPVPLVVADRCAKRGHDVYVANLREKHVVVNSIKEANELLVNQGGLKKLRYNA

SEQ ID NO: 6 - protein, 910 aa, Angiostrongylus costaricensis
MDQLEGAMRNLTVKTVAMPAKRSPGKRGVKSEFLTNLTKLTLKPNVPFFKYDVRMYVVYKGKDGGEHLKELTKQTKDDFPEQERKASSVIVYKHLIKSYPDVFTKDGALFYDRAAILFSAQVQLKFDGEKKEFHLPASVLSSVDDNAGVRVIIKKVTDSFQVTSNDIVKAVNVRDIERDKSILEVLNLAVSQEGYLETLKFVAYGSTVQYLFDHQAFGFRDDELPELMDGKYVGIGLSKSVKVLEGDDGKCSPFVVADVTKSAFHVDEQNLLEKISQMSIFFDYETGKSNFSVQTASRPNVMKDILRLVKGLYVKTLYGKTRTFPIGGISAAASSLRFQATDGKQYTVEQYYMKQYNIQLKYPGLFTVSERHNPQTYYPVELLAVAPSQRVTQQQQTPSDVAALIRASATLPLHRLNQTGVMKKALKIAPGNFLLEAAGISVDKNFTKVVGRVLPSPTILYGGSSITKVDVCCKWSWDRAHFLKPANLSNWAVCVMLSQNDFRRVQINEFIGRVEVRCRSHGMQVSPASQVSYLKRQTYDGLKEWYAEQKKKNRKYLLFITSDNIQQHDTIKLLEVHYQIVSQEVKAGTVNAVVLKNQNQTLDNVISKINQKIGGVNYNIVLDSGLSDNVRNWLSDNGVLFVGFEISNPSALSRMEIARGATNKMPSVLGWGANCSSNPQQYMGDYVYVEPRQSDMIGSKLAEIVIEIIKRHRMTTRVTLHHIIFYFSGISEGQFSMISDIYMRAVYTGISCLSLQNVLKVTALVVSKDHNERIYKSNIVGKRAADQNIPGGTVVDTVIVSPVINEFYLNAHSAFQGTAKTPKYSLVADNSQISLDAIEGITYGLCYLHEIVTATVSLPVPLIVADRCAKRGHSVYVANLREKHVVVNSIQEANELLVNQGDLRKLRYNA

SEQ ID NO: 7 - protein, 908 aa, Dictyocaulus viviparus
MDQLRNAMGDLNMKTVAVPEKRPSGNRGMKTEFLTNITKLSLKPNVPFFKYDVRMYVVYKGTDGREHLKELTKQTKDDFPEQQRKSLAVLVYKHLIKSYPDVFLKGVALFYDRASVLFSAQRQIKLAAEKEEFIVPASILSNACGDAEKVCVVIKKVSDSFQVTSNDIMKAVNVREFERDKNILEVLNLAVSQEGYLETTKFVVYGSNEHYLYDHRAYGFRDNELPELMDGKYMGIGLSKSVKVLEGENGKCGPFFVADVTKSAFHADEQNLLDKISQMSVFLDRRTGAYNFSVDVACKPYNMKNILQLLKGLYVKTSYGKTRTFPIGNLAPAANILRFQASDGNQYTVEQYFKKHYNIKLKYPSLFTVSERHNPHTYYPVELLFVAPSQRVTSQQQTPEDVAAIIKASATLPQHRLNQTKVMKDALKMVPGNTYLEAAGITVDKDFAKVVGRVLPAPTVVYGRSETIAVNECKWNWDRSQFIQPGNLSNWAVCATLTPNDSGRIKIREYISRVESRCRSHGMKIEAAAEIFYLKRQTFEELKEWYAAQKKKNRKYLMFITSDSIKQHDLIKLLEVQYQIVSQEVKASKVDAVMFKNQNQTLDNVVAKINEKIGGVNYNITLNTAAGDNNCLSDSGVLFIGFEISNPPSSSKTEVIICTAHKIPSVLGWGANCASNPQQFIGDYVYVEARQSDMMGSKLAGVVMDIIERHRLATKTAIRHLIFYFSGISEGQFGMIPDSYMRAINTGLSSLSPQCVACVTALAVSKDHNERIYRANITGKRAADQNIPGGTVVDTKIVSPVMNEFYLNAHSAFQGTARTPKYSLLADNSMMPLDVIEGLTYGLCYLHEIVSATVSVPAPLIVADRCAKRGHNIYLANMKEKQVVVGTIKEANEKLVNRGDLKKVRYNA

SEQ ID NO: 8 - protein, 913 aa, Haemonchus contortus
MADQLGAGMKQLSVSTVALPEKRDPGSRGTKTEYLTNLTNINLKPNVPFYKYDVRMYIVYKGNDGREHLKELTKQTKDDFPEQERKIAAVGVFKYMCKNYKDVFPNDGALFYDRAAVLFSAQNELKLGGQERTLSIKPTANLVPAAGKDAAEVRVVIKKVTEGYQVTSNDLIKAVNIRDCERDKGMLEVLNIALSQKGYLETSQFVTYGTGVHYLFDHRALGFRDNELPSLMDGKYMGIGLTKSVKVLEGDGKKGSAYVVTDVTKGAFHIDEQSLLEKISQMSIFFDPRRGQSTFNVQAAMQPFNMKSILQLIKGLYVRTIYGKKKCFPIGNIASPANQISFETESGKATVEQYFKKHYNIQLKYPTLFTVSERHNPSTYYPVELLVVAPSQRVTLQQQTPDQVASMIKASATLPAVRIQQTKVMKDALGITSGNAKLSSAGISVEDSFTKVPGRVLPAPTIIYGGNQGIRPKDNCKWNGDQSKFIEPAQLTNWAVCATLTQNDSRRLKIGDYIARVEARCRLRGMQVERAAEIFDLKKQTFEGLREFYAAQKQKDRKYLMFITSDSIKQHDYIKLLEVEYQIVSQEVKGSKVDSVMFKNQNQTLDNVIAKINMKLGGVNYVVSLGPRMDDPVSKWLNDEARLFVGFEISNPPALSKMEIERGATYKMPSVLGWGANCAANPQHYLGDYKFIKARQSDMMGATLGELIVEILKKFKAATSRAPRHIVLYFSGISEGQFSLVTDTYVRAIRTGIKSLSEAYNPNVTALAVSKDHNERLYKSKIVGERATDQNIPPGTVVDTKIVSPVINEFYLNAHSAFQGTAKTPKYSLLADDSNIPLDVIESMTHGLCYLHEIVTSTVSVPVPLIVADRCAKRGHNIFIANSGQGKAAVSSIEEANERLTNHGELQKVRYNA

SEQ ID NO: 9 - protein, 912 aa, Ancylostoma canium
MADQLKKAMGDLTVSTIALPEKRPSGTTGANTEFVANLTSLKLKPNVPFYKYDMRMYIVYKGNDGKEHLKELTKQTKDDFPEQERKTGTVLVYKHLLKSYPNIFPQDGALLYDRAAVLFSAQKQIKLDGDEKVFTLPASLVPSAGEDAVGVRVVIKKVTDGFQVTSNDLQKVVNVRDMEKDKGILEVLSLAMSQKGYMETSQFVTYGSGVHYLFDHRALGFKDHEVPELMDGKYMGVGLSKSVKVLEGEKGQPCGAYVVTDVTKGAFHMDDQNLLEKISQMSMFIDPRSGQSHFSVQAVTQPFNQKNILQLIKGLYVRTTYGKKKTFPIGNLAQPANQLKFQTTDGTQCTVEQYFKKHYNIQLKYPGMFTVSERHNPHTYYPVELLRVAPSQRVTLQQQTPDQVAAMIKACATLPQNRLHQTKLLKDALAIKEGNPHLSAAGISVVNGFTSVPGRVLPSPSIVYGGNQLVKPVDNCKWNGDRSRFLEPARLHNWAVCATLTQNDSRRLNVKEYVAKIEGRCRQRGMDVEPCAEIFNLQRQNFESLKEWYASQKAKNRRYLMFLTSDGIKQHDLIKLLEIEYQIVSQEIKGSKVDAVVSRNQNQTLDNVVAKINEKLGGVNYNIMLGARPTDDVNKWISDKDRMFVGFEISNPPALSKVEIERGATYRMPSVLGWGANCAKNPQQYLGDYVYIEPRQSDMMGAKLSELIVTILKRFRAATDVAPRHIVLYFSGISEGQWSMVADTYMRAIHTGIKSLSASYKPSLTALTVSKDHNERIYKANITGNRATEQNIPPGTVVDTKIVSPVINEFYLNAHSAFQGTAKTPKYALVYDDSNIPMNVVEGMTHGLCYLHEIVTATVSVPVPLIVADRCAKRGHNVYIANSNQRDAVGSIKEANERLVNQGELQKVRYNA

SEQ ID NO: 10 - protein, 913 aa, Nippostrongylus brasiliensis
MVDQLQGRVEKLSVSNVALPEKRAPGSAGVKQDFVCNLTALKLKPNVPFYKYDIRIYVVFEKSDGTEHQKELTKQTKDDFPEQERKTASVYVFKTLCKMYKDIFPQDSALFYDRAAVLFSAQTQLKLGGEEKQFKLPAKGVPSTGADAKGILVVIKKVTDKFQLTSNDLMKAVNVRDCEREKEILEVLTLAVSQKGYLETSQFVTYGSGVHYLFDHRALGFRDNELPELMDGKYMGIGVTKSVKVLEGEDGTGATPFVVTDVTKGAFHIDDQNLMEKISQMSIFFDQRTGQSSFSQRTAMQPHNQKTILQQIKGLYVRTTYGAKPRTFPIGNIARPASELKFEAADGSTFTVEQYFKKQYNIQLKYPWMFTVSERHKPTSYYPVELLRVAPSQRVTLQQQTPDQVASMIKASATLPQTRLHQTKVMKDALDINSRNPYLASAGITVNNDFATVTGRILPTPTILYGERQEVKPVDNCKWNGDRSRFLEPAYLSNWAVCATLTQFDARKIQVKDYVSRIEARCRNRGMQVDPVAEIFHLQNQSFEGLKEWYAAQKQKGRRYLMFITSDGIKQHDLIKLLEIEYQIVSQEVKGSKVDAVLHRNQNQTLDNVVAKINMKLGGVNYNVMLGRPNEPSSKWLTDKNRLFIGFEISNPPPLSKAEMERGATYKMPSVLGWGANCASNPQQYIGDYLYIQARQSDMMGQKLSEVVVDILKRFREATSVAPQHMVLYFSGISEGQFSLVTDTYMKAIHTGISSLSANYKPHITALAVSKDHNERIYKSKISGTRASEQNIPPGTVVDTKIVSPVINEFYLNAHSAFQGTAKTPKYSLLADDSKVPLDIVERMTHGLCFLHEIVTSTVSVPVPLIVADRCAKRGHNVFIANSGQGRSAVGSIDEANAKLVNHGELQKVRFNA

SEQ ID NO: 11 - protein, 912 aa, Oesophagostomum dentatum
MADQLKKAMGELSVSTVALPEKRPPGTMGARTEFVANVTSLKLKPNIPFFKYDIRMYVVYKGADGKEHLKELTKQTKDDFPEQERKNATVLVYKSLVKNNSKVFPPEGALFYDRAAVLFSAGTQIKLDGDEKQFMMPASLVPSAGEDAVGVRVVIKKVTEGFQVTSNDLAKAVNVRDIEKDKGLLEVLNLAMSQKGYLETSQFVTYGSGVHYLFDHRALGFRDQEVPELMDGKYMGIGLTKAVKVLEGDKGQSCGAFVVTDVTKGAFHMDDQNLLEKISQMSMFIDPRSGQSHFNVQSAMQPFNQKAILQLIKGLYVRTTYGKKRTFPIGNIAQPASQLKFQTVDGKQCTVEQYFKQHYNIQLKYPAMFTVSERHNPHTYYPVELLRVAPSQRVTLQQQTPDQVATMIRACATLPQNRLQQTRVLKDALGIKDGNPHLSAAGISVVNGFTSVPGRVLPSPSIVYGGNQLAKPIDNCKWNGDRYRFLEPASLRNWAVCVTLTPNDSRRLHVKDYVARIEGRCRQRGMEVEPCSEIFTLQRQNFDSLKEWYVSQKAKNRRYLMFITSDNIKQHDLIKLLEIEYQIVSQEVKGSKVDAVLTKNQNQTLDNVVAKVNQKLGGVNYNVMLGANPNEAVNKWISEKDRMFVGFEISNPPALSKVELERGGTYKMPSVLGWGANCAKNPQQYLGDYLYIEPRQTDMMGAKLSELIVQILKRFRGATEVAPRHIVLYFSGISEGQWSMVADTYVRAIQTGIKSLSPNYKPNLTALTVSKDHNERLYRANITGNRASEQNVPPGTVVDTKIVSPVINEFYLNAHSAFQGTAKTPKYALVYDDSHIPIDVVEGMTHGLCYLHEIVTATVSVPVPLIVADRCAKRGHNVYIANSNQRNAVGSIDEANARLVNQGELQKVRYNA

SEQ ID NO: 12 - protein, 912 aa, Ancylostoma ceylonicum
MADQLKKAMGDLTVSTIALPEKRPPGTTGANTEFVANLTSLKLKPNVPFYKYDIRMYIVYKGKDGKEHLKELTKQTKDDFPEQERKTGTVLVYKHLLKSHPNIFPQDGALLYDRAAVLFSAQKQIKLDGDEKVFTLPANLVPSAGEDAVGVRVVVKKVTDGFQVTSNDLQKAVNVRDIEKDKGILEVLSLAMSQKGYMETSQFVTYGSGVHYLFDHRALGFKDHEVPELMDGKYMGIGLSKSVKVLEGEKGQPCGAYVVTDVTKGAFHMDDQNLLEKISQMSMFIDPRSGQSHFSVQAATQPFNQKNILQLIKGLYVRTTYGKKKTFPIGNLAQPANQLKFQTTDGTQCTVEQYFKKHYNIQLKYPGMFTVSERHNPHTYYPVELLRVAPSQRVTLQQQTPDQVATMIKACATLPQNRLHQTKLLKDALAIKEGNPHLSAAGISVVNGFTSVPGRVLPSPSIVYGGNQLVKPVDNCKWNGDRSRFLEPARLHNWAVCATLTQNDSRRLNVKEYVARIEGRCRQRGVDVEPCAEIFNLQRQNFESLKEWYASQKAKNRRYLMFLTSDGIKQHDLIKLLEIEYQIVSQEIKGSKVDAVLSRNQNQTLDNVVAKINEKLGGVNYNIMLGTRPTDDVNKWISDKDRMFVGFEISNPPALSKVEIERGATYRMPSVLGWGANCAKNPQQYLGDYVYIEPRQSDMMGAKLSELIVQILKRFRSATDVAPRHIVLYFSGISEGQWSLVADTYMRAIHTGIKSLSASYKPSLTALTVSKDHNERIYKANITGSRATEQNIPPGTVVDTKIVSPVINEFYLNAHSAFQGTAKTPKYALVYDDSNIPMNVVEGMTHGLCYLHEIVTATVSVPVPLIVADRCAKRGHNVYIANSNQRDAVGSIKEANERLVNQGELQKVRYNA

SEQ ID NO: 13 - protein, 718 aa, Acanthocheilonema viteae
MGQTSITGPPVCLTEKIAPGTRKIELLDVVTNVWGLIPHENIPIYRYDFRVMEEYPPKKGSPLLKEVTKQTKNDYLTVDRKTKCMVIYQILLKREEQFFGTLDSLIYDRASTLYSLRKLPFSKDEKEEIFFVKPNELPMNIVGENCIMVHVHIKPCKDDFQLTMNDLSSCVSNNPDQINRSLQQFLEILAMQEVFFMEGRFVSYGTGECYLMDPNQFGFGERDVPELQEGKYVAIGAAKGVRIIEGPRGKKGINAALVIDVKKAAFHIDNQNLFKKAEDILRKSVDLTRRIDQQSITVLNKALKGLYVRCNYGKNRAFVIAGVSKENARTSKLVTKDGEMSVEKYFGMKYSMKLKYPFLPLIIERFPPKNNFYPIEVLSVCENQRVSKGQQTSFQVQTMVKACAIPPSLRLQQTNVLSKAMNLGTFDRNRWMEKCNVAVTNNLELKARVLPMPAIEYRTNGWVKPSEKTSWEDGKNQYLIPAVCKKWCAVALMGPRERLNEYQFRNYIRTFLQHCRRHGMEMDDPFLCEYIQRSKQEDIEPLITRAKHSGAVFIHFITADELNYHAHMKYVESQEQIVTQDLKASTAVAVVTQNKRQTLDNIVNKTNVKLGGMNYSVHLETNCDIWLTKPGLLIVGLDIAHPVFSNVSKRDRNSIPSVVGYSANIKKHPLDFIGGYRYGKANMEEMADDTIQHIFSDILRYFNANRGKPPTHLFVIRD

SEQ ID NO: 14 - protein, 911 aa, Anisakis simplex
MEINCSKMELTDESAEERLVMPSKKISGMRLKAADSIAVVMNGYEIDLSNVPEKVYKHELKLMAVKQDNQLRDLTRGPRNDVSITLRRRVLWSVYTTVLKNYKDFFGSDTKMYFYDCGVTLYSINKILREDGEKEFRLKMDQLSSNSRDFLGVKVVGIVAKLLACEEVYLRDFDESISECIDERQRSLQQFLEVAINQKLYHDQDHLLFGNKAYHRAMPHDKQLKGGKLLQSGFAKNIRVVGDSVENAVIVAQLDAKKSAFFKEQNLVALIASLCNNRFTLLNDANTRRRVAKQLKGLVVRTNHLPKSQRIFSIFGMTKESASKVIIVIDDEEISIEEYYNHKYQIGLQYTNLPCIVERRFQAKNYYPMECLDVCKGQRVENKKQTPDLVEELIAGCRLLPNRLKEENEKQRRAASITNQNPYFRRLGMLYPPAIIYGNNDRVEPNSNGHWRLNRQHLFYSPANPPRRWVVFIFEDAVSKELFDRFLYSYIERAQSHGIKLQRPSRIETIDRVDMDYLMDKMKMMRKNCVEYVMFITKDKRDPVHDKMKLTEVQASVVTQHIFSGTIQRSIGNRGAEMTLDNLIMKMNLKLGGISHAIAASTPFMRCNHLNEDICKRIWLRPTRMFIGLDMSHSSPLSFYERQAGFHASEPTVVGMAYTCGSEFGMRGLYWMQEPRVYTIQSLKEHLVEALNQFKEENKSYPEHVIVFRGGISDGQFQKVMTLEANAFRAAFEALPSSTPPNKIRLSLICVQTNSNYRLLLDNLQVDGNALQQNVPSGTCVDSSIVHPTQAEFILVAHKSIMGTARPIRCTVLVDDPRMGLDEVEGITNCLCYMHGIVTSPISVPAHLYAASNLAKRGRNNWKIVCNNGDDDVSIASGDGSHNQFHNDGAPDFFTNISSHLAPKLKHKFWA

SEQ ID NO: 15 - protein, 907 aa, Ascaris Lumbricoides
MPIAVQDMPVRFAEKVSPGRRHAETVDLISNVWGVIPKKNVPIYRYDVRILEEFPPKASGAPTKEVTKQCRDDFPSIERKNRCVAVFLRLLEREEAFFGKRESVVYDRASILYTLDRLQIENDETKTFIVTPNELPEGSVSTDCVRVLFNIKQCTEDFQLTTSDLKQGVSLEGERINRSLQQFFELLASQEAFFTEGRFVTYGTGQSFLFEPYDFGFRDQDMPPLPDGKYIGIGASKGVKLIEGPSGPGGIHAALVMDVKKAAFHIEQQSVAEKVAMIFNVLSMSVDPRQIPQLKKLLKGLYVRCEYGKQRVFMITNIATQNALQMRFRCDDMMVTVADYFASKYDIRLKYPQLPLVIERRPSGESYYPMEKLIVCENQRVTQTQQSSAQVQAMIKACATLPIHRIRQTTAMTRAMKLDGTELNRWMREYSVNVTKNLTLKGRVLPPPRIEYGRNERTIVNPERTSWLANRNHYLLPAKCEKWHVVALVGPSERFSNDKLRAYVRAFMNQCRNRGMQMADPMVVDYVRGAREQEVDVRMQKAKQMGATFVHFVTSDMLKFHGHIKLVEMQLQIVTQDLTTRTASQAPQKWQTLDNIVNKTNLKLGGINFGLILENEIFRFSAQKWLMNEGRLVVGIDVAHPPLAAVRGIDRTKVPSVVGYSSNCKKFPLEFIGGYRYATANMEELTDNSIRDVIVDSIRKFQVNRGKLPDHLFILRDGISEGQYKYVVVSEVEGVKKACGLVGGIGYRPNITYIVATKLHNMRLFKKNINQQDKATGQNIKPGTVVDKHIVNPVLNEFYLNSHSAFQGTAKTPRYTILFDTAKVPSDEIQAIVYALAYNHQIVNAAISLPAPIVIAARMASRGRSNYAVQFGEGSDSTEGGRERNIAELNANMGYMDKPLSDCRFNA

SEQ ID NO: 16 - protein, 929 aa, Ascaris suum
MLMKTDQGNHLITSVMAEVEELARRAERMEIQDMPVRFADKVSPGRRHAETVDLISNVWGVIPKKNVPIYRYDVRILEEFPPKASGAPTKEVTKQCRDDFPSIERKNRCVAVFLRLLEREKAFFGKRESVVYDRASILYTLDRLQIENDETKTFIVTPNELPEGSVSTDCVRVLFNIKQCTEDFQLTTSDLKQGVSLEGERINRSLQQFFELLASQEAFFTEGRFVTYGTGQSFLFEPYDFGFRDQDMPPLPDGKYIGIGASKGVKLIEGPSGPGGIHAALVMDVKKAAFHIEQQSVAEKVAMIFNVLSMSVDPRQIPQLKKLLKGLYVRCEYGKQRVFMITNIATQNALQMRFRCDDMMVTVADYFASKYDIRLKYPQLPLVIERRPSGESYYPMEKLIVCENQRVTQTQQSSAQVQAMIKACATLPIHRIRQTTAMTRAMKLDGTELNRWMREYSVNVTKNLTLKGRVLPPPQIEYGRNERTIVNPERTSWLANRNHYLLPAKCEKWHVVALVGPSERFSNDKLRAYVRAFMNQCRNRGMQMADPMVVDYVRGAREQEVDVRMQKAKQMGATFVHFVTSDMLKFHGHIKLVEMQLQIVTQDLTTRTASQAPQKWQTLDNIVNKTNLKLGGINFGLILENEIAQKWLMNEGRLVVGIDVAHPPLAAVRGIDRTKVPSVVGYSSNCKKFPLEFIGGYRYATANMEELTDNSIRDVIVDSIRKFQVNRGKLPDHLFILRDGISEGQYKYVVVSEVEGVKKACGLVGGIGYRPNITYIVATKLHNMRLFKKNINQQDKATGQNIKPGTVVDKHIVNPVLNEFYLNSHSAFQGTAKTPRYTILFDTAKVPSDEIQAIVYALAYNHQIVNAAISLPAPIVIAARMASRGRSNYAVQFGEGSDSTEGGRERNIAELNANMGYMDKPLSDCRFNA

SEQ ID NO: 17 - protein, 923 aa, Brugia malayi
MAGSSAVEKVRMQMEQASVSRLPVRLADKIAPGIRKSELLDVVTNVWGLISHENIPIYRYDFRVLEEYPPKKDSPSFKEVTKQVRNDYVAVDRRAKCFAVYQALLRREKQFFGAADTLIYDRASILYSLRKLSFPRDEVRKTFFLNKNELPANIVSDDCVKVHVNIKPCKEDFQLAMHDLKNCVSNNPDEINSSLQQFLEILAMQEVFFMQGRFVSYGTGECYLMDPSQFGFGDRDMPELQEGKYVAIGAAKGVRIIEGLGNKRGINAGLVLDAKKAAFHIDNQHLLEKVESIFRRRAEIGHGIDQQSILILSKALKGLYVRCSYGKNREFAIAGISKENAHTSKLALKTGEMSVEKYFMTKYSIKLKYPKLPLIMERCQPKNNLYPIEVLVVCENQRVSKGQQTSSQVQTMIRACATEPSVRLMQTNRLSQAMKLDNSNQHKWIGKCNMTITDNFMFPARVLPQPVIEYHTNGWINPNEKTVWMDGKKYYLIPAVCEKWYAVALMGPRERFHENQFCTYIKMFLERCRKHGMQIRDPIGYEYIRRSKEQDIESLIIKAKKSGATFIHFVTADELNYHARMKYIESQEQILTQDLKASTALGIVMKRQYQTLDNIVNKTNIKMGGLNYSVHLEENCDRWLGRPGFLIVGLDIAHPSYSRVPNKDRNTVLSVVGYSANIKKHPLDFIGGYRFAKAQMEELMDDAIQQIFSDLLRYFNANRGTPPTHLFVIRDGISVGQYKYVMNTEVEQIKHACQLVGGQNYRPHITFIVLTKMHNLRIYKKNIHKQERAAQQNIKPGTVIDKHVVNPVLSEFYLNSHSTFQGTAKTPRYTLLFDTSKMEADEMQGIVHALAYNFQIVNMAVSLPSPVMIASRMAKRGRCNYVAMFGDGSESSDNGKNNEKDAVELNKQLSYINKPLEVVRFNA

SEQ ID NO: 18 - protein, 922 aa, Dirofilaria immitis
MAGCSGVEKVRTQLERTSVSEPPVYLTEKIAPGSRAYESLDVVTNVWGLIPHKNIFVYRYDFRLFEEYPPKGRSPVMKEVTKQTRNDYLSVDRKTKCLTIYQTLLKRQKQFFGDVHSLIYDRASILYSLRQLPFPKDEQRVAFFLNPNELPEDMVAEDCVKIHVYVKPCKEDFHLSMKDLKSGVSNNPDEINRNLQQFLEILTMQEVFFMEGRFISYGSGECYLLEPDQFGFGERDVPELREGKYVAVGASKGVRIIEGPQEGGMNAALIIDVKKAAFHIDNQSLLEKIEHILGKRGDLMRGIDQQSIAILNKALKGLYVLCNYGKKRAFMVAGISKKNARTSKLITKSGEVSVEEYFKMKYSMKLKYPAMPLVMERCQPKNNFYPIEVLSVCVNQRVSIGQQTSSQVQTMIRACAAVPSLRLHQTNTLSKAMKLDNAKGNRWITNCSVGITNNLAFSARVLPAPAIEYGVNGWIKPDEKTSWKVEKNQYLIPAICKNWYAAALMGPRERMNENLFRNYIRIFLQHCREHGMEMGDPLGCEYIRRAKQQDVEPLIAKAKSLGATFIHFVLADELNYHAHIKYIESQEQIVTQDLKATTALAVTLQHKRQTLDNIVNKTNIKMGGLNYSVHLETNCDQWITKPQFLIVGLDIAHPAISMVSKRDQNIVPSVVGYSANIKKHPLDFIGGYRYAKAQMEELVDDTIQQIFSDLLRYFNANRGKPPMHLFIIRDGVSIGQYKYIMNIEVEQIKKACQMVGGPGFRPHITFIVLTKMHGLRIYKKNINKQEGSAKQNIKPGTIIDKYVVNPVINEFYLNSHSTFQGTTKIPRYTLLFDTSEMKADVMQGIVHALAFDFQIVNMAVSRPSPVMIASRMAKRGRCNYVAMYGDEMENSDNGKGIEKDLVELNNKLSYVSKPLEAVRFNA

SEQ ID NO: 19 - protein, 908 aa, Enterobius vermicularis
MSHIDVECVEKQVGELKLRSRVLLPGKTSPGQRCEGRIRVQTNVFGIKPLEDVPFFRYDFRVLEEYPSKKQDPVFKEVTKQTRNDYVSIERKNKCVAVYLMVMHQEANFFGDIGAFVYDRASTLYSLDKLKLADGEVKSFTVVARSLPKDLFSEDCLRVIVKVKQCAQEFQLSSMDLKSGFNLNPDLVSRSLQQFYELLLSQDAFFTEGRFVCYGSGENYLYNPLDFGFSAQETPGLPDGKYIGVGAIKGVKIVEGSQDRPTLSFSVDVKKAAFHIELQSIAEKVSDICNCVGVRLDDRHVKMLSKMLKGLYVRCNYGKRRTFIISGISEQSVDSFKLEKDGKMIGMARYFMDKYKIALKFGKLPLVVEKTSRGTHYYPMELLFVCENQRVNLSQQSSRQVQQMIRACATVPSMRKRQMLDMVSALNLDGGRKNRWCREFQVHLAERTYELEARVLQKPTIVYGGDGRVEMKESGAWTITQNTAYLLPSVCERWIIIALVSASDRFSRNDLENYTALFLERCRSRGISIRKPMEVIHIPRAREQDVDIAFKNAEKMSARFIHFVTSENLKYHEKIKFLESQYQILTQDLMTKNAAVIRKRPQTLDNIVHKTNLKLGGINFDLHLESEEAQKWIGRKDRLIIGMDLTFTGVPSKDKSSKSPSVIGYAMNCHAHPLDFTGGYRFCWTINEEVGDKTFCDIVSESLQLAKKNRRVPVHLVILRDGVSEGQYKYAIEKEVQDVKDACARVGGAKYKPYITFVLATKRHQVRIYRSKIDPSRRAVDQNIPPGTVVDTEVVNPVYNEFYLNSHAAIQGTAKTPRYNVLYDNSSMSSDEVQGMVYALTFNMQIVNQPCSLPAPLIIADQMAARGRSNFIAFCGRSSSSVGSLDLDHINSELGYMGKELSNYRFNA

SEQ ID NO: 20 - protein, 922 aa, Iitomosoides sigmodontis
MAAGFDVEKITVQMGQASVREPPIYLAEKVPPGTRKFESLDVVTNVLGLIPQKNIPVYRYDFCVLEEFPPKKGSPFFKEVSKQTKNDYLTVDRKTKCLLVYQALLKREKQFFGNMDSLIYDRASILYSLRKLPMPKDEQRVTFFVMPDELPVNIVCEDCIKIHTHVKPCKEDFQLTMSDLKSCVSNNLDNINRSLQQFLEILVMQEVFFMEGRFVSYGTGECYLMDPNQFGFGDRDAPLLEEGKYVAIGAAKGVRIIEGPRGKEGINAALVFDVKKAAFHIDNQNLLEKVEHILRKRVDLTRGIGQQSITVLNNALKGLYVRCDYGKRRAFIIAGVSKENARTSKLVTKDGEMTIEKYFETKYSMKLKYPTLPLIMEKCQPKSNFYPIEVLSVCENQRVSKGQQTSSQVQTMIRACATLPSIRLQQTNVLSKAMKLDNSNRNKWMDKCNVTVTSNLEFKARVLPMPAIEYRTNGWVKPCEKTSWTDGRNQYLIPAVCKNWCAVALMGQRERFNEHQFRNYIQIFLKHCRRHGMEMGDPLVLEYIHRSKQQDIESLVLKAKRLGATFIHFVTADELSYHAHMKYIESQEQIVTQDLKASTAIAVAMQNKAQTLDNIVNKTNIKMGGLNYSVHLETDCDGWLMKTGLLIVGLDIAHPVWMSSRKDQNGVPSVVGYSANIKKHPLDFIGGYRYGKANAEEMVDDTIQHIFADVLQYFNANRGKPPVHLFVIRDGISVGQYKYVMKYEVENIKKACQMVGGPGFRPHITFIVLTKMHRLRIYKKNIRKQDRGAEQNIKPGTVIDEYVVDPTINEFYLNSHSAFQGTTKTPRYTLLFDTSGMKADVMQGIVYALAYDFQIVNMAVSRPSPVMIASRMAKRGRSNYMAMYGDDSENSDNGNGIEKDLVELNNRLSYISKPLEVVRFNA

SEQ ID NO: 21 - protein, 746 aa, Loa loa
MNDLKSCVSNNPDEINHSLQQFLEILAMQEVFFMEGRFVSYGAGECYLMYPNQFGFGERDTPELEEGKYVAVGAAKGVRIVEGPRGEGGINAALVIDVKKAAFHVDNQCLLEKVECILRRRVILMRGIDHLSIAILSKALKGLFVRCNYGKNRAFTIGGVSKENARTSKLVSRTGEMSVEKYFEMKYSVKLKYPTLPLIMERCQTKSNFYPMEVLIVCENQRVSKGQQTPSQVQTMIRACATVPSLRLQQTNTLSQAMKLNSSNQNKWMAKCNVAVTNNLTFTARVLPTPSIEYRTNGWIKPSEKTSWTVGKYQYLIPGVCRNWYAVALMGPRERFNEHQFRKYMDIFLQHCRLHGMEMRDPLKYVYIPHAKQQNVEPLITEAKSLGATFIHFVTADELNYHAHIKYIESQEQVVTQDLKASTALSVTTQNKRQTLDNIVNKTNIKLGGLNYSVHLETNCDRWLTKTGLLIVGLDIAHPYFAVPRKDRSCVPSVIGYSANIKKHPLDFIGGYRYGKAEVEELTDDTIQQIFSDILRYFNANRGEPPKHLFVIRDGISTGQYKYVMNTEVEQIKKACQMVGGPGFRPHITFIVLTKMHSLRIYRKNIRKQERAVEQNIKPGTIVDKHVVNPVLNEFYLNSHSAAQGTTKTPRYTLLFDTSGMEADVMQGIVHALAYNFQIVNMAVSLPCPVMIAARMAKRGRCNYIAMYGDESENSDNGSGVEKDLIELNNQLSYISKPLEAVRFNA

SEQ ID NO: 22 - protein, 615 aa, Onchocerca flexuosa
MFFPRGRFVSFGSGETYLMDPSQFGFSERDMPELEGGKYIAIGASKGVRIIEGPQEGGINAAMVIDVKKAAFHADNQDLLEKVECLLRKRSDLKRGVDQQSIAILNKALKGLYVLCNYGKKRAFTVTGVSKENARTSKLVTKSGEMSVEKYFEMKYSMKLKYPTLPLIMERSQPKNNFYPIEVLSVCENQRVSKGQQTSSQVQTMIRACATVPSLRLQQTNALSKAMKLDATGENKWMKNCSVVVTNNLMFPARVLPSPDIEYRINGWVKPSEKTSWLTGKNQYLIPAVCNKWYAVALIGPRERMNENLFRNYIRIFLQHCRQHGMEMSEPLGCEYIRRANQQDIEPLIIKAKNLGATFIHFVTADELNYHGHMKYIESKEQVVTQDLKATTAVAVAVQNKRQTLENIVNKTNIKLGGLNYSVHLETNCDKWLTKAGFLVVGLDIAHPAVSMVSRKDRNFVPSVIGYSANIKKHPLDFIGGYRYCKAEMEELVDDTMQEVFSYILRYYKASRGEPPNHLFVIRDGVSAGQYKYVMNTEVQQIKKACQMVGGPNFCPHITFIVLTKMHNVRLYKKNIHKQERPAEQNIKPGTIIDKHVVNPVLNEFYLNSHLAFQV

SEQ ID NO: 23 - protein, 657 aa, Onchocerca ochengi
MHVIREKMKKAAFHMDNQDLLEKVECVLRKRIDLKRGVDQQSIAVLSRALKGLYVLCNYGKKRAFTVAGVSKENARTSKLVTKSGEMSVEKYFEMKYSMKLKYPTLPLIMERCQPKNNFYPIEVLSVCENQRVSKGQQTSSQVQTMIRACATAPSLRLQQTNALSKAMKLDATGENRWMKNCNVVVTNNLTFPARVLPTPAIEYRINGWVKPNEKTSWMTGRNQYLIPAVCNKWYAVALMGPRERMNENLFRNYIRIFLQDCRQHGMQMSEPLGCEYIRRANQQDVEPLIIKAKNLGATFIHFVTADELNYHGYMKYIESREQVVTQDLKATTAVAVAMQNKRQTLENIVNKANIKLGGLNYSVHLETNCDNWLTKAGFLIVGLDIAHPAVSMVSKKDRNIVPSVIGYSANIKKHPLDFIGGYRYGKAEMEELVDDTMQEIFSYILRYYKTSRGEPPKHLFVIRDGVSAGQYKYVMNTEVQQIKKACQMVGGPNFCPHITFIVLTKMHNVRLYKKNIRKQERPAEQNIKPGTIIDKHVVNPVLNEFYLNSHSAFQGTTKIPRYTLLYDTSDMKADVMQGIVHALTYNFQIVNMAVSCPSPVMIAARMAKRGRCNYVAMYGDESENSDNGSGIEKDLTQLNNQLSYISKPLEAVRFNA

SEQ ID NO: 24 - protein, 923 aa, Onchocerca volvulus
MAAKYSDVEKVRMELGQASVSELPVRLAKKVAPGTRACESLDIMTNVWGLISRENIPVYRYDFRVLEEYPPKKGTPAFKEVTKQTRNDYLAVDRKTKCLAIYQTLLKHQKQFFGDMYSLIYDRASILYSLRKLPIPEDERRVEFFLKPNELPKNTVADDCLQVHVFVKPCKEDFQLTMNDLKSGVSNDPDKINRSLQQFLEILAMQEVFFMQGRFVSFGSGETYLMDPSQFGFGERDMPELEEGKYIAIGASKGVRIIEGQREGGINAALVIDVKKAAFHIDNQDLLEKVECVLRKRIDLKRGVDQQSIAVLSRALKGLYVLCNYGKKRAFTVAGVSKENARTSKLVTKSGEMSVEKYFEMKYSMKLKYPTLPLIMERCQPKNNFYPIEVLSVCENQRVSKGQQTSSQVQTMIRACATAPSLRLQQTNALSKAMKLDATGENRWMKNCNVVVTNNLTFPARVLPTPAIEYRINGWVKPNEKTSWMTGRNQYLIPAVCNKWYAVALMGPRERMNENLFRNYIRIFLQDCRQHGMQMSEPLGCEYIRRANQQDVEPLIIKAKNLGATFIHFVTADELNYHGYMKYIESREQVVTQDLKATTAVAVAVQNKRQTLENIVNKANIKLGGLNYSVHLETNCDNWLMKAGFLIVGLDIAHPAVSMVSKKDRNIVPSVIGYSANIKKHPLDFIGGYRYGKAEMEELVDDTMQEIFSYILRYYKTSRGEPPKHLFVIRDGVSAGQYKYVMNTEVQQIKKACQMVGGPNFCPHITFIVLTKMHNVRLYKKNIRKQERPAEQNIKPGTIIDKHVVNPVLNEFYLNSHSAFQGTTKIPRYTLLYDTSDMKADVMQGIVHALTYNFQIVNMAVSCPSPVMIAARMAKRGRCNYVAMYGDESENSDNGSGIEKDLTQLNNQLSYISKPLEAVRFNA

SEQ ID NO: 25 - protein, 897 aa, Syphacia muris
MDQDTNDLVKKVAAMKVNDLQFATKRSEGLESQAKCVVASNIWEIRLLKDVPFYRYEICVLEEYPTKDEKHAFKELTKKTRDDYQTVERKNKCVMVFETLLQKERAFFGNAMSYVYDMASIVFSLNKLKIDGEKKFSIERGSLPEGLFPLDCIQVIVSVKPCVEDFVITAKECFSHSYLLKQFQALVQVYDLLLSQEAFFSKDVFVSYGASESYLLEPWNFGFDKGLPDFPGGKYVGVGCCKAVRVIEGQGAPTLSVCLDVKKAIFHKDCQNLVEKTTEICKSGINNLEAWQVKNLEHFLKGLHVRCCYGKFRTFSIKGLSTGNAEKSRVQIREKSVSIVQYYKEKYNKVLQYKHLPLVIEKTSKGYNYYPMELLEVCENQRVHNAQQLPVEAQQLIRACAIPPSERLRQIACIARAMKLNNGKSNSWLKEFGLEISKKNLQVTARVIRKPNVRYGMGAVFEIEDKGEWKMSPNTELLIPAACDLWCVYALIANSDTFNHCDLKSYVQFFLDRCQKRGMKMKNPVEVALLHRPAEADLEKCFQLANKSHVKFIHFITTQSLPIHGRMKYLELKYRIITQDVLSKTAIAVKRKPLTLDNIINKVNVKLGGMNFELFPASLNFKTWFLSNEKLVISYKLSPVHLKTGKSVKSLSVIGLSSNCQKHPQKFSCGYRYTHSNKDGETAVCDIVAESLLLVKKNRQIPDHIIIFRSGIPSAAYDYALKKEVVDIEEGCSKAFGKSYQPKITFVVVTTAHNVRLFRPESSGKGRTFDQNVPVGTVVDRQLVSNQLTEFYLNSHAAMQGTSKIPRYDVLFDTFGMSSDAIQSLAFTLCFDLQIVNHACSLPAPVMIARDMATRGENDISIFWQSWLKGSESCDIQCLNEQLGHMSKPLADLFLTT

SEQ ID NO: 26 - protein, 927 aa, Thelazia callipaeda
FSQRIRDSEEVKHESKRIADRKIKPSSTCLAQKVAPGTRAGEYIEVVTNVWGMIPQKNIPIFRYDFRVLEEYPPKKGSPAMKEVTKQTRNDYIAVERKSKCVIVYQTLLKREKQFFGPPDSIVYDRAAILYSLKKLPITGDESQITFYLDPKELPANTVGDECVKIHVNVKPCKDDFQLTMNDLKNSVSNNPDEVNRSLQQFLEILAMQEVFFTHGRFVSYGTGQCYLLDPDQFGFKAQDMPELQEGKYVAIGASKGVRVIEGPKGSGGINAALVIDVKKAAFHIDNQDLVEKAGNILKRKTELNRGLDQQSIAVLNKSLKGLYVRCGYGKERTFAIGGISKDTPRSLKLVTKTGEMSVEQYFFKKYAIRLKYATLPCIMERSQPRNKFYPMEVLSVCENQRVSQGQQTSNQVQAMIRACATLPSRRISQTAALTEAMKLRGQIGNRWLKGCDVTITDTLTLTARVLPAPSIEYSTNGRLKPNEKTSWSVGNNHYLIPATCKNWFAVALMGPRDKLNETQFRTYIRTFLLHCRQHGMEMSEPLGCEYIYRTKQQDVESLISKAKRMGATFIHFVTADELDYHAQMKYVESQEQILTQDLKASTAAAVVLQNKWQTLDNIINKTNVKLGGLNYSVHLEKNCDDWLLKPGLLVVGLDIAHPPVSAVLGKDRHVVPSVIGYSANIKKHPLDFIGGYRYGKAEMEELMDESIHDIFVDIMRYFKNNRGKPPTHLFVIRDGVSEGQYKYVLNTEIEQIKKACNTVGGSFYRPHITFTVLTKMHSIRLFKKNIAHQAKAVEQNIKPGTVIDKHVVNPVLNEFYLNSHSAFQGTTKAPRYTLLFDSSKMSADEMQGIVHALAYDFQIVNMAVSRPSPVMIASRMAKRGRSNYIAMYGDDDSQHSSGSAVEADLVNLNNQLSYISKPLEGVRFNA

SEQ ID NO: 27 - protein, 790 aa, Wuchereria bancrofti
MFLSYVAVDRKMKCFAVYQALLKREKQFFGAVDTLIYDRASTLYSLRKLSFPRDEVRKTFFLNKNELPMNIVGGDCVRVHVNIKPCKEDFQLSTHDLKNCVSNNPDEINNSLQQFLEILAMQEVFFMEGRFVSYGTGECYLMDPSQFGFGDRDMPELQEGKYIAIGAAKGVRIIEGLGGKRGISAGLVLDAKKAAFHIDNQDLLEKVESIFRRRADLSRGIDQQSILILSKALKGLYVRCTYGKNREFVIAGISKENARTSKLVHKTGEMSVENYFVMKYSIKLKYPILPLIMERCQPKNNLYPIEVLVVCENQRVSKGQQTSSQVQTMIRACATDPSVRLKQTNILSKAMKLDNSNQHKWMGKCNMTITDNLMFPARVLPQPTIEYHTNGWVNPNEKTVWMDGRNYYLIPAVCEKWYAIALMGPRERLREDQFGTYIKMFLERCRKHGMQIKDPIGYEYIRRAKEQDVESLMIKAKKSGATFIHFVTADELNYHANIRYFSCDKWLGRPGFLIVGLDIAHPAYSMLPNKDRNSVLSVVGYSANIKKHPLDFIGGYRFAKAEMEELIDDTMQQIFSDLLRYFNANRGKPPTHLFVIRDGISVGQYKYVMNTEVEQIKQACQSVGGRNYRPHITFIVLTKMHNLRIYKKNIHKQERAAQQNIKPGTIVDKHVVNPVLSEFYLNSHSTFQGTTKIPRYTLLFDTSRMEADEMQGIVHALAYNFQIVNMAVSLPSPVMIASRMAKRGRCNYVAMFGDESENSDNGRGVEKDVVELNNQLSYISKPLEVVRFNA